(19) United States
(12) Patent Application Publication
(10) Pub. No.: US 2026/0159815 A1
(43) Pub. Date: Jun. 11, 2026

(54) METHODS OF CULTURING CARDIAC CELLS AND USE OF CELLS FOR DISEASE MODELING

(71) Applicants: Technion Research & Development Foundation Limited, Haifa (IL); Rambam Med-Tech Ltd., Haifa (IL)

(72) Inventors: Oren CASPI, Haifa (IL); Idan Refael HAIM, Haifa (IL); Noam KAZMA, Haifa (IL)

(73) Assignees: Technion Research & Development Foundation Limited, Haifa (IL); Rambam Med-Tech Ltd., Haifa (IL)

(21) Appl. No.: 19/390,823

(22) Filed: Nov. 17, 2025

Related U.S. Application Data
(63) Continuation of application No. PCT/IL2024/050488, filed on May 17, 2024.
(60) Provisional application No. 63/467,014, filed on May 17, 2023.

(51) Int. Cl. C12N 5/071; C12N 5/00; G01N 33/50

(52) U.S. Cl. CPC C12N 5/0697; C12N 5/0018; G01N 33/5023; G01N 33/5026; G01N 33/5088; C12N 2500/34; C12N 2501/2301; C12N 2501/24; C12N 2501/32; C12N 2501/33; C12N 2501/365; C12N 2501/727; C12N 2502/1323; C12N 2502/1329; C12N 2502/1347; C12N 2502/28; C12N 2503/02; C12N 2506/45; C12N 2513/00; G01N 2500/10

(57) ABSTRACT

An isolated multicellular organoid comprising cardiomyocytes, endothelial cells, fibroblasts and smooth muscle cells is disclosed. The endothelial cells form part of a 3D endothelial structure, the cardiomyocytes of the organoid are capable of synchronized contractions, and epicardial cells of the organoid do not form a distinct layer in the organoid. Methods of maturing immature cardiomyocytes and use of cardiomyocytes for generating disease models are also disclosed.

### RELATED APPLICATIONS

This application is a Continuation of PCT Patent Application No. PCT/IL2024/050488 having International filing date of May 17, 2024 which claims the benefit of priority of U.S. Provisional Patent Application No. 63/467,014, filed on May 17, 2023. The contents of the above applications are all incorporated by reference as if fully set forth herein in their entirety.

### FIELD AND BACKGROUND OF THE INVENTION

The present invention, in some embodiments thereof, relates to methods of culturing cardiac cells and, formation of cardiac organoids therefrom.

The ability to accurately detect toxicity and efficacy, particularly towards the heart, remains a considerable challenge in the development of new drugs. Models of human heart disease are needed to develop effective therapies, and existing models are imperfect, as shown by the frequency with which compounds showing promise in a model system fail to exhibit efficacy in clinical trials.

Due to intrinsic fundamental differences in the physiology of the human heart compared to traditional experimental animal models, researchers have focused efforts on developing in vitro models using cardiomyocytes derived from human pluripotent stem cells that can better predict the human-specific response to a compound of interest (Mercola, M., Circ. Res. 112 (2013) 534-548).

While many such in vitro human stem cell models have been developed over the years, most have focused on simple designs that minimally recapitulate the functions of the heart. The models have limited outputs that can be insufficient in studying a response to a compound of interest. For example, a monolayer of cardiomyocytes can be useful in studying cardiac electrophysiology but is limited in the ability to study inotropic agents that require a measure of contractile force. Simple 3D models such as cardiac tissue strips can be used to study changes in developed force but lack outputs that are more clinically relevant such as pressure-volume relationship, ejection fraction, and stroke work. The lack of physiological relevance also restricts the capability of the models to recapitulate many disease phenotypes needed for efficacy testing, especially since heart diseases are often inextricably linked to the hemodynamic loading conditions on the heart. Thus, there is a need for more biologically complex cardiomimetic human in vitro models that can provide additional insights in both drug development and disease modeling (Passier, R., et al. Cell Stem Cell 18, (2016) 309-321).

Heart failure with preserved ejection fraction (HFpEF) is a growing epidemic affecting over 1% of the population. Nevertheless, there are almost no therapies capable of ameliorating disease advancement or improving survival. The paucity of models for HFpEF, specifically those based on human tissues, may contribute to the lack of a disease modifying therapeutic option. A major reason for that is the multifactorial nature of the disease, in which various comorbidities such as obesity, hypertension, and diabetes mellitus type 2 lead to the development of HFpEF.

### SUMMARY OF THE INVENTION

According to an aspect of some embodiments of the present invention there is provided an isolated multicellular organoid comprising cardiomyocytes, endothelial cells, fibroblasts and smooth muscle cells, wherein the endothelial cells form part of a 3D endothelial structure, the cardiomyocytes of the organoid being capable of synchronized contractions, wherein epicardial cells of the organoid do not form a distinct layer in the organoid.

According to some embodiments of the invention, the smooth muscle cells and/or the fibroblasts are distributed evenly throughout the organoid.

According to some embodiments of the invention, the isolated multicellular organoid is devoid of a chamber.

According to some embodiments of the invention, the cardiomyocytes, the endothelial cells, the fibroblasts and the smooth muscle cells have an identical MHC presentation.

According to some embodiments of the invention, the organoid is not comprised on, or in, a scaffold.

According to some embodiments of the invention, the organoid is devoid of immune cells.

According to some embodiments of the invention, the organoid comprises immune cells.

According to some embodiments of the invention, the organoid is devoid of blood vessels of greater than 10 μm.

According to some embodiments of the invention, the fibroblasts and the endothelial cells are derived ex vivo from human pluripotent stem cells via the formation of endocardial cells.

According to some embodiments of the invention, the cardiomyocytes are derived ex vivo from human pluripotent stem cells.

According to some embodiments of the invention, the smooth muscle cells are derived ex vivo from human pluripotent stem cells.

According to some embodiments of the invention, the human pluripotent stem cells are induced pluripotent stem cells (iPSCs).

According to some embodiments of the invention, the iPSCs are generated from dermal fibroblasts of a healthy human subject.

According to some embodiments of the invention, the human pluripotent stem cells are embryonic stem cells.

According to some embodiments of the invention, the 3D endothelial structure comprises a lumen.

According to some embodiments of the invention, at least 40% of the cells of the organoid comprise cardiomyocytes.

According to some embodiments of the invention, a surface area of the multicellular organoid is greater than 3 mm².

According to an aspect of some embodiments of the present invention there is provided a method of generating a multicellular organoid comprising:
(a) culturing a first population of pluripotent stem cells under conditions that promote differentiation of the pluripotent stem cells into cardiomyocytes;
(b) culturing a second population of pluripotent stem cells under conditions that promote differentiation of the pluripotent stem cells into endothelial cells;
(c) culturing a third population of pluripotent stem cells under conditions that promote differentiation of the pluripotent stem cells into fibroblasts;
(d) culturing a fourth population of pluripotent stem cells under conditions that promote differentiation of the pluripotent stem cells into smooth muscle cells; and subsequently
e) combining the cardiomyocytes, the endothelial cells, the fibroblasts and the smooth muscle cells under conditions that promote formation of the multicellular organoid.

According to some embodiments of the invention, the pluripotent stem cells are human pluripotent stem cells.

According to some embodiments of the invention, the pluripotent stem cells comprise induced pluripotent stem cells (iPSCs).

According to some embodiments of the invention, the iPSCs are derived from human fibroblasts of a healthy subject.

According to some embodiments of the invention, the conditions that promote formation of the multicellular organoid comprise insulin depravation and a culture period of at least 10 days.

According to some embodiments of the invention, the conditions that promote formation of the multicellular organoid comprise:
(a) culturing the cardiomyocytes, the endothelial cells, the fibroblasts and the smooth muscle cells in a medium being substantially devoid of insulin for at least two days;
(b) supplementing the medium with VEGF to generate a supplemented medium; and
(c) culturing the cardiomyocytes, the endothelial cells, the fibroblasts and the smooth muscle cells in the supplemented medium for at least 7 additional days.

According to some embodiments of the invention, the conditions for culturing the third population of pluripotent stem cells comprise a first set of conditions for differentiating the pluripotent stem cells into epicardial cells and a second set of conditions for differentiating the epicardial cells into the fibroblasts.

According to some embodiments of the invention, the conditions for culturing the fourth population of pluripotent stem cells comprise a first set of conditions for differentiating the pluripotent stem cells into epicardial cells and a second set of conditions for differentiating the epicardial cells into the smooth muscle cells.

According to some embodiments of the invention, the method further comprises contacting the multicellular organoid in a maturation medium comprising:
(i) an mTOR inhibitor; and
(ii) free fatty acids;
under conditions that promote maturation of the multicellular organoid, wherein an expression of TNNI3 in a mature multicellular organoid is at least 1.5 fold higher than an expression of TNNT2 in the mature multicellular organoid, as measured by RT-PCR.

According to some embodiments of the invention, the free fatty acids are selected from the group consisting of linoleic acid, palmitic acid, propionic acid, arachidonic acid and linolenic acid.

According to some embodiments of the invention, the maturation medium further comprises a steroid hormone and a thyroid hormone.

According to some embodiments of the invention, the steroid hormone comprises dexamethasone.

According to some embodiments of the invention, a concentration of the mTOR inhibitor in the medium is between 1 nM and 100 nM.

According to some embodiments of the invention, a concentration of the free fatty acids in the medium is between 0.1%-5%.

According to some embodiments of the invention, the amount of glucose in the medium is between 1 mM-20 mM.

According to an aspect of some embodiments of the present invention there is provided a multicellular organoid obtainable by the methods described herein.

According to an aspect of some embodiments of the present invention there is provided a method of obtaining mature cardiomyocytes comprising:
(a) differentiating pluripotent stem cells into immature cardiomyocytes, wherein an abundance of TNNI1 or TNNI2 in an immature cardiomyocyte is at least 1.5 fold higher than an abundance of TNNT3 in the immature cardiomyocyte, as measured by Western Blot; and
(b) culturing the immature cardiomyocytes in a maturation medium comprising:
(i) an mTOR inhibitor; and
(ii) free fatty acids;
under conditions that promote maturation of the immature cardiomyocytes, wherein the protein abundance of TNNI3 is detectable and upregulated as measured by Western blot analysis.

According to some embodiments of the invention, the prime metabolic substrate for the mature cardiomyocyte is fatty acids, and a prime metabolic substrate for the immature cardiomyocyte is glucose or lactate.

According to some embodiments of the invention, the mTOR inhibitor comprises torin-1.

According to some embodiments of the invention, the maturation medium further comprises a steroid hormone and a thyroid hormone.

According to some embodiments of the invention, the steroid hormone comprises dexamethasone.

According to some embodiments of the invention, the concentration of the mTOR inhibitor in the medium is between 1 nM and 100 nM.

According to some embodiments of the invention, the concentration of the free fatty acids in the medium is between 0.1%-5%.

According to some embodiments of the invention, the amount of glucose in the medium is between 1 mM-20 mM.

According to some embodiments of the invention, the immature cardiomyocytes are comprised in a multi-cellular organoid.

According to some embodiments of the invention, the immature cardiomyocytes are comprised in a uni-cellular organoid.

According to some embodiments of the invention, the multi-cellular organoid comprises the cardiomyocytes, endothelial cells, fibroblasts and smooth muscle cells.

According to an aspect of some embodiments of the present invention there is provided a method of generating a cell model for Heart failure with preserved ejection fraction (HFpEF) comprising:
culturing human cardiomyocytes in a medium comprising at least one hypertension-associated hormone and at least one metabolic disease promoting agent, under conditions that bring about at least one of the following phenotypes:
(i) an increase in expression of collagen in the cardiomyocytes as compared to prior to the culturing;
(ii) an increase in relaxation time of the cardiomyocytes (e.g. by at least 20%) as compared to prior to the culturing;
(iii) an increase in production of NT-proBNP by the cardiomyocytes as compared to prior to the culturing; or
(iv) an increase in cardiomyocyte size (e.g. by at least 20%) as compared to prior to the culturing.

According to some embodiments of the invention, the conditions bring about each of the phenotypes.

According to some embodiments of the invention, the cardiomyocytes are comprised in a cardiac organoid.

According to some embodiments of the invention, the cardiac organoid is a unicellular cardiac organoid.

According to some embodiments of the invention, the cardiac organoid is a multicellular cardiac organoid.

According to some embodiments of the invention, the multicellular cardiac organoid comprises endothelial cells, fibroblasts and smooth muscle cells.

According to some embodiments of the invention, the human cardiomyocytes are derived ex vivo from pluripotent stem cells.

According to some embodiments of the invention, the pluripotent stem cells comprise iPSCs.

According to some embodiments of the invention, the human cardiomyocytes are mature cardiomyocytes.

According to some embodiments of the invention, the method further comprises maturing immature cardiomyocytes to generate the mature cardiomyocytes prior to the culturing.

According to some embodiments of the invention, the maturing comprises culturing the immature cardiomyocytes in a medium comprising:
(i) an mTOR inhibitor; and
(ii) free fatty acids.

According to some embodiments of the invention, the mTOR inhibitor comprises torin-1.

According to some embodiments of the invention, the at least one hypertension-associated hormone comprises Angiotensin-II and/or Endothelin-1.

According to some embodiments of the invention, the metabolic disease promoting agent comprises an obesity promoting agent and/or a diabetes promoting agent.

According to some embodiments of the invention, the obesity promoting agent comprises fatty acids and inflammatory cytokines.

According to some embodiments of the invention, the inflammatory cytokines are selected from the group consisting of IL-1β, IFN-γ, TNF-α and interleukin 1α.

According to some embodiments of the invention, the diabetes promoting agent comprises glucose at a concentration above about 10 mM.

According to some embodiments of the invention, the at least one hypertension-associated hormone comprises Angiotensin-II and/or Endothelin-1 and the at least one metabolic disease promoting agent comprises fatty acids, IL-1β, IFN-γ and glucose at a concentration above about 10 mM, and wherein the conditions comprise insulin depravation.

According to an aspect of some embodiments of the present invention there is provided an HFpEF cell model generated according to the methods described herein.

According to an aspect of some embodiments of the present invention there is provided a method of determining whether an active agent is useful for treating HFpEF comprising:
(a) contacting the agent with the HFpEF cell model described herein,
(b) measuring at least one of the following characteristics of the cardiomyocytes:
(i) expression of collagen;
(ii) relaxation time;
(iii) production of NT-proBNP;
wherein a decrease in expression of collagen in the cardiomyocytes, a decrease in relaxation time, and/or a decrease in production of NT-proBNP as compared to prior to the contacting is indicative of an agent useful for treating HFpEF.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C illustrate morphology of multicellular micro-organoids. (A) Immunostaining of multicellular micro-organoids for CMs and EC specific markers, blue-DAPI, red-α-actinin, green=CD31 in the presence and absence (control) of maturation conditions (B) H&E stain of multicellular micro-organoid. (C) RT-qPCR of ECs and SMCs marker genes (CADH5 [Cadherin 5], SMTN [Smoothelin]). Control is unicellular organoids.

FIGS. 2A-C. (A) Representative immunostained images showing cardiomyocytes treated with (left column) or without (right column) maturation media (11.1 mM glucose), blue=DAPI, red=α-actinin. (B) RT-qPCR of selected sarcomere genes (TNNI1 [troponin I1], TNNI3 [troponin 13, cardiac type], MYH6 [myosin heavy chain 6], MYH7 [myosin heavy chain 7], TNNT2 [troponin T2, cardiac type]), iPSC-derived cardiomyocytes following treatment with maturation media. (C) Western blot analysis of ssTnI and cTnI in low and high glucose maturation media.

FIG. 3 is a bar graph illustrating results of RT-qPCR of selected sarcomere genes (TNNI1 [troponin I1], TNNI3 [troponin 13, cardiac type]) after treatments with different Torin1 concentrations (10 nM and 25 nM) and different medias (maturation media (3 mM glucose) and RPMI-B27), control group treated with RPMI-B27.

FIGS. 4A-B. (A) ATP test for ECs after 7 days treatment with maturation media 3 mM and 11.1 mM glucose, control group treated with M199. (B) Tube formation assay for ECs after 7 days treatment with maturation media 3 mM and 11.1 mM glucose, control group treated with M199, compared to HUVECs.

FIGS. 5A-C. (A-B) Force development and dynamics using unicellular micro-organoids (cardiomyocyte only). Analysis with musclemotion-ImageJ macro. (*P<0.005, **P<0.0001). (C) unicellular micro-organoid area calculated with ImageJ.

FIGS. 6A-E. (A) Multicellular micro-organoids area calculated with ImageJ (P<0.0001). (B-E) Force development and dynamics using multicellular micro-organoids. Analysis with musclemotion-ImageJ macro (P<0.0001).

FIGS. 7A-E: Ultrastructural analysis of micro-organoids. A. Bright-field images of the micro-organoids, during relaxation. Scale-bar: 100 μm B. Immunofluorescent staining of micro-organoid, cTnT in red, DAPI in blue. Scale-bar: 20 μm. C. Size differences as an index of hypertrophy. Measured as the difference between exposed micro-organoids and their pre-induction size. D. Masson-Trichrome staining of micro-organoids. Arrows pointing at collagen deposits, Scale-bar: 50 μm. E. Fibrosis percentage out of total micro-organoids size. Measured using image processing techniques. Data presented as mean±SEM. *P<0.05, **P<0.01, ***P<0.001, ****P<0.0001.

FIGS. 8A-D: Contraction dynamics analysis of micro-organoids. analyzing relative changes between exposed micro-organoids and their pre-exposure measurements (1 week/basal). A. Relaxation time B. Contraction amplitude. C. Time to peak. D. Contraction duration. Data presented as median and IQR. *P<0.05.

FIGS. 9A-D: Assessment of restrictive features in HFpEF. A. The settings of the experiment. On the upper left, representative pictures of the ring-shaped hCO. On the upper right, a force transducer coupled with the length controller used in the experiment. The lower graph presents a representative force-length experiment, paced in 1 Hz and stretched in a series of growing lengths up to a 0.8 mm stretch. B. The passive force of the organoids, measured as the force (mN) applied on each 0.1 mm stretch. C. Maximal passive force (mN) measured following a 0.8 mm stretch. Median and IQR are presented. D. NT-proBNP content (pgr/ml) in the media of the exposed organoids. Data presented as mean±SEM. *P<0.05, **P<0.01.

FIGS. 10A-D: Diastolic evaluation of HFpEF induction. A. Relaxation constant τ (tau) of the hCOs. (msec) B. Representative calcium transients of single contraction cycles, from 0% (baseline) to 100% (peak). C. Time from peak-down of the calcium transients (msec). D. Real-time PCR of ATTP2A gene, encoding to SERCA, presenting relative expression, normalized to GAPDH. Data presented as mean±SEM. *P<0.05.

FIGS. 11A-D: Oxidative stress and energetics evaluation. A. Reactive oxygen species (ROS) content, measured as the fluorescent of the ROS indicator B. Real-time PCR of oxidative stress related genes: Xbox binding protein1-spliced (XBP1s), Xbox binding protein1-unspliced (XBPlu), and NOS2. Presenting relative expression, normalized to GAPDH. C. Mitochondrial activity, measured by the basal oxygen consumption rate, OCR (pmol/min). D. ATP content, measured by the fluorescence of the ATP indicator. Data presented as mean±SEM. *P<0.05, **P<0.01, ***P<0.001, ****P<0.0001.

### DESCRIPTION OF SPECIFIC EMBODIMENTS OF THE INVENTION

The present invention, in some embodiments thereof, relates to methods of culturing cardiac cells and, formation of cardiac organoids therefrom.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cardiac organoids are in vitro self-organizing and three-dimensional structures composed of multiple cardiac cells (i.e., cardiomyocytes, endothelial cells, cardiac fibroblasts, etc.) with or without biological scaffolds. Since cardiac organoids recapitulate structural and functional characteristics of the native heart to a higher degree compared to the conventional two-dimensional culture systems, their applications, in combination with pluripotent stem cell technologies, are being widely expanded for the investigation of cardiogenesis, cardiac disease modeling, drug screening and development, and regenerative medicine.

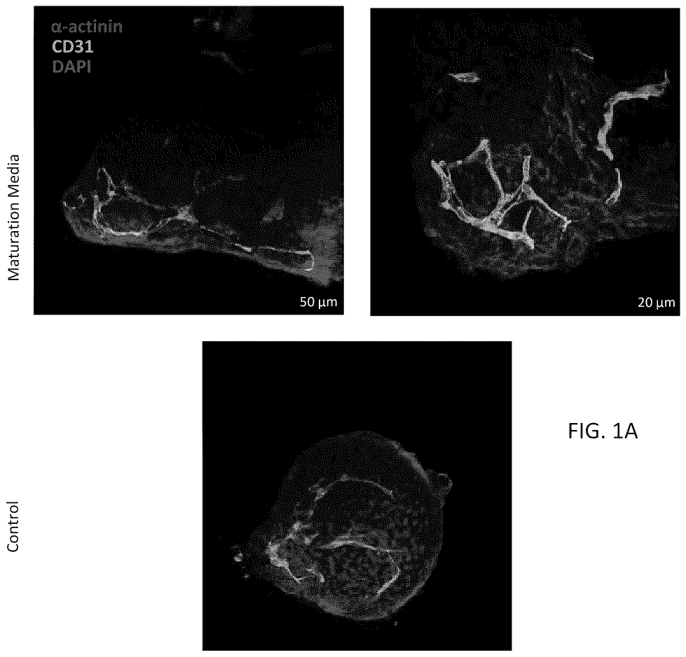

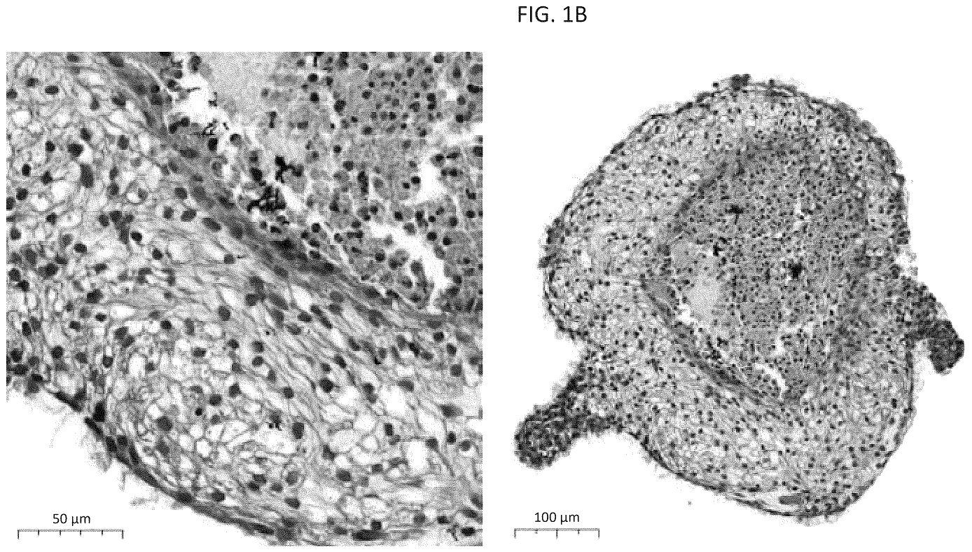

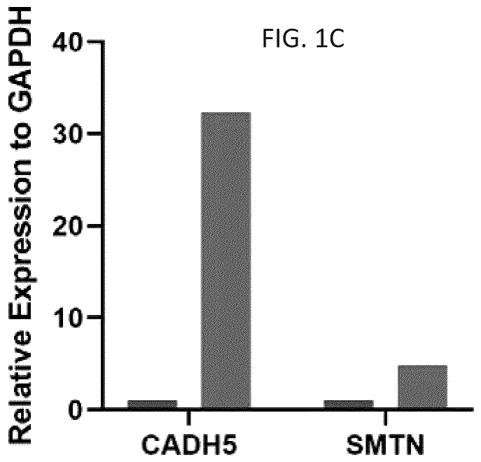

The present inventors have now generated a 3D cardiac organoid from a plurality of cells, each differentiated ex vivo from pluripotent stem cells. The organoids were shown to comprise cardiomyoctes capable of synchronized contractions, endothelial cells and smooth muscles cells, as detected by immunofluorescence and RT-PCR. The organoids developed vessel-like structures which demonstrates the functional interplay between the endothelial cells and the other supporting cells (FIGS. 1A-C).

According to a first aspect of the present invention there is provided an isolated multicellular organoid comprising cardiomyocytes, endothelial cells, fibroblasts and smooth muscle cells, wherein the endothelial cells form part of a 3D endothelial structure, the cardiomyocytes of the organoid being capable of synchronized contractions, wherein epicardial cells thereof (e.g. smooth muscle cells and/or the fibroblasts) do not form a distinct layer in the organoid.

The term “organoid” as used herein, refers to a 3D structure comprising a collection of cell types which are present in a corresponding natural organ.

The organoid may be of any shape/dimension. Typically, a surface area of the multicellular organoid is greater than 3 mm² and more preferably greater than 5 mm².

The term “multi-cellular” refers to the organoid comprising a plurality of cell types—including cardiomyocytes, endothelial cells, fibroblast cells and smooth muscle cells.

In one embodiment, at least some of the cells of the organoid are human cells.

In another embodiment, all of the cells of the organoid are human cells.

In one embodiment, the organoids of the present invention consist of cellular material—for example devoid of synthetic scaffold.

In other embodiments, the organoids of the present invention are devoid of synthetic or non-synthetic scaffolds.

In one embodiment, epicardial cells are cells originating from epicardial tissue and include fibroblasts and smooth muscle cells (coronary vascular smooth muscle cells). In another embodiment, epicardial cells further include pericytes.

As used herein, the term “cardiomyocytes” refers to fully or at least partially differentiated cardiomyocytes.

According to embodiments, the organoid of the invention fulfills at least one functional phenotype specific to cardiac tissue (e.g. appropriate response to a chronotropic agent and/or ability to spontaneously contract in a synchronized function).

According to one embodiment the cardiomyocytes of the present invention are at least capable of spontaneous contraction.

In another embodiment, the cardiomyocytes comprises gap junctions.

According to another embodiment, the cardiomyocytes of the present invention express at least one marker (more preferably at least two markers and even more preferably at least three markers) of early-immature cardiomyocytes (e.g. atrial natriuretic factor (ANF), Nkx2.5, MEF2C and α-skeletal actin).

According to another embodiment, the cardiomyocytes of the present invention express at least one marker (more preferably at least two markers and even more preferably at least three markers) of fully differentiated cardiomyocytes (e.g. MLC-2V, α-MHC, α-cardiac actin and Troponin I).

Screening of partially differentiated cardiomyocytes may be performed by a method enabling detection of at least one characteristic associated with a cardiac phenotype, as described herein below, for example via detection of cardiac specific mechanical contraction, detection of cardiac specific structures, detection of cardiac specific proteins, detection of cardiac specific RNAs, detection of cardiac specific electrical activity, and detection of cardiac specific changes in the intracellular concentration of a physiological ion.

Various techniques can be used to detect each of cardiac specific mechanical contraction, cardiac specific structures, cardiac specific proteins, cardiac specific RNAs, cardiac specific electrical activity, and cardiac specific changes in the intracellular concentration of a physiological ion. For example, detection of cardiac specific mechanical contraction may be effected visually using an optical microscope. Alternately, such detection can be effected and recorded using a microscope equipped with a suitable automated motion detection system. Detection of cardiac specific structures may be performed via light microscopy, fluorescence affinity labeling and fluorescence microscopy, or electron microscopy, depending on the type of structure whose detection is desired. Detection of cardiac specific proteins may be effected via fluorescence affinity labeling and fluorescence microscopy. Alternately, techniques such as Western immunoblotting or hybridization micro arrays (“protein chips”) may be employed. Detection of cardiac specific RNAs is preferably effected using RT-PCR. Alternately, other commonly used methods, such as hybridization microarray (“RNA chip”) or Northern blotting, may be employed. RT-PCR can be used to detect cardiac specific RNAs. Detection of cardiac specific changes in the intracellular concentration of a physiological ion, such as calcium, is preferably effected using assays based on fluorescent ion binding dyes such as the fura-2 calcium binding dye (for example, refer to Brixius, K. et al., 1997. J Appl Physiol. 83:652). Such assays can be advantageously used to detect changes in the intracellular concentration of calcium ions, such as calcium transients. Detection of cardiac specific electrical activity of the cells may be effected by monitoring the electrical activity thereof via a multielectrode array. Suitable multielectrode arrays may be obtained from Multi Channel Systems, Reutlingen, Germany. To detect cardiac specific electrical activity in the partially differentiated cells, the latter can be advantageously cultured, under conditions suitable for inducing cardiac differentiation directly on a multielectrode array, thereby conveniently enabling monitoring the electrical activity of such cells and tissues.

According to a particular embodiment, the cardiomyocytes are mature cardiomyocytes.

According to another embodiment, the cardiomyocytes are immature cardiomyoctes.

The present inventors contemplate organoids, wherein at least 40%, 45%, 50%, 55% or even 60% of the cellular material thereof are cardiomyocytes.

According to a particular embodiment, the cardiomyocytes are differentiated ex vivo from pluripotent stem cells (e.g. embryonic stem cells and/or induced pluripotent stem cells).

Mature cardiomyocytes may be distinguished from immature cardiomyocytes by their ultra-structural, molecular, metabolic, and electrophysiological signature. Unlike mature, adult cardiomyocytes, the immature cardiomyocytes differentiated from human induced pluripotent stem cells (hiPSC-CMs) lack the rod-shaped morphology characteristic of adult cardiomyocytes, and most are mononuclear. The myofibrils of the hiPSC-CMs are chaotically organized, have a shorter sarcomere length (1.65 μm vs. 2.2 μm), and lack the A, H, and M bands on electron microscopy. The isoform profile of the proteins mediating the contractile machinery is significantly different between adult cardiomyocytes and hiPSC-CMs.

While hiPSC-CMs predominantly express myosin heavy chain 6 (MYH6), myosin light chain 2 atrial isoform (MLC2A), and slow skeletal-type troponin I (ssTnI) encoded by TNNI1, adult cardiomyocyte predominantly express myosin heavy chain 7 (MYH7), myosin light chain 2 ventricular isoform (MLC2V), and cardiac troponin I (cTnI) encoded by TNNI3, respectively. The metabolism of hiPSC-CMs is significantly different than those of adult cardiomyocytes and resembles that of the failing myocardium. The main metabolic substrate of hiPSC-CMs is glucose or lactate (vs. fatty acid oxidation for adult cardiomyocytes). The electrophysiological signature of the hiPSC-CMs is significantly different than that of adult cardiomyocytes. In addition, hiPSC-CMs display low sarcoplasmic reticulum (SR) calcium content, lower maximum contractile force, slower upstroke velocity, higher resting potential, and spontaneous beating due to the presence of the pacemaker current I_{f} and reduced densities of the hyperpolarizing current I_{K1}.

Endothelial cells may be human embryonic stem cell (hESC)-derived endothelial cells (see for example, Levenberg, et al., Proc Natl Acad Sci USA (2002) 99, 4391-4396, the contents of which are incorporated by reference herein), induced pluripotent stem cells derived endothelial cells (see for example Fan et al. Int J Mol Sci. 2022 August; 23 (15): 8507, the contents of which are incorporated herein by reference) or primary endothelial cells cultured from e.g. human umbilical vein (HUVEC), or biopsy-derived endothelial cells such as from the aorta or umbilical artery. The endothelial cells of the present invention may also be derived from humans (either autologous or non-autologous) e.g. from the blood or bone marrow. In addition the endothelial cells may be derived from other mammals, for example, humans, mice or cows. For example, endothelial cells may be retrieved from bovine aortic tissue.

In one embodiment, human embryonic stem cell derived endothelial cells are produced by culturing human embryonic stem cells in the absence of LIF and bFGF to stimulate formation of embryonic bodies, and isolating PECAM1 positive cells from the population. HUVEC may be isolated from tissue according to methods known to those skilled in the art or purchased from cell culture laboratories such as Cambrex Biosciences or Cell Essentials.

In one embodiment, human endothelial cells are differentiated from induced pluripotent stem cells via the formation of endocardial cells (see Example 1, herein below).

In one embodiment, the organoids of the invention are vascularized i.e. there is formation of at least a part of a 3D blood vessel network in the cardiac tissue. Typically, the blood vessel network is comprised of endothelial cells. The vasculature may be at any stage of formation. In one embodiment the organoid comprises at least one 3D endothelial structure. Examples of 3D endothelial structures include, but are not limited to tube-like structures, such as those comprising a lumen.

Fibroblasts may be isolated from tissue according to methods known to those skilled in the art (e.g. obtained from E-13 ICR embryos) or purchased from cell culture laboratories such as Cambrex Biosciences or Cell Essentials. In one embodiment, fibroblasts are differentiated from induced pluripotent stem cells via the formation of epicardial cells (see Example 1, herein below). Other exemplary methods for differentiating fibroblasts from hiPSCs and embryonic stem cells are disclosed for example in Shamis Y, et al. (2013) PLOS ONE 8 (12): e83755. doi: 10.1371/journal.pone.0083755.

Smooth muscle cells may be obtained by ex vivo differentiation of pluripotent stem cells (see for example Markou et al., Front Bioeng Biotechnol. 2020; 8:278 and Example 1 herein below).

As mentioned, epicardial cells of the organoid do not form a distinct layer (e.g. outer layer) in the organoid i.e. at least the fibroblasts and smooth muscle cells of the organoid are not organized into a distinct layer. Rather, they are found throughout the organoid typically in the vicinity of the endothelial cell structure. This is in sharp contrast to the naturally occurring cardiac tissue which is made up of three different layers, the epicardium, myocardium, and endocardium. In nature, the fibroblasts and smooth muscle cells differentiate from the epicardial layer and migrate inwards into the tissue. In sharp contrast, the present invention differentiates smooth muscle cells and fibroblasts ex vivo via formation of epicardial tissue, such that these cells are distributed throughout the organoid.

As mentioned, the cells of the isolated cardiac organoid are typically generated ex vivo from pluripotent stem cells—e.g. induced pluripotent stem cells (iPSCs) or embryonic stem cells (ESCs).

According to a particular embodiment, each of the cells of the organoid are derived from human induced pluripotent stem cells (hiPSCs). The same source of cells may be used to generate the iPSCs. For example, a cell sample derived from a single subject (e.g. dermal fibroblasts) may be used to generate iPSCs, which subsequently can be differentiated ex vivo into each of the four cell types of the organoid. Thus, it is contemplated that each cell of the organoid presents with an identical MHC presentation.

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin or blood cells) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc/LIN28 in a somatic stem cell.

According to one embodiment the method is effected by expressing in the cells at least one polypeptide belonging to the Oct family or the Sox family.

According to another embodiment, the method is effected by expressing in the cells at least two polypeptides—one belonging to the Oct family and one to the Sox family.

Examples of polypeptides belonging to the Oct family include, for example, Oct3/4 (NM_013633, mouse and NM_002701, human), Oct1A (NM_198934, mouse and NM_002697, human), Oct6 (NM_011141, mouse and NM_002699, human), and the like. Oct3/4 is a transcription factor belonging to the POU family, and is reported as a marker of undifferentiated cells (Okamoto et al., Cell 60:461-72, 1990). Oct3/4 is also reported to participate in the maintenance of pluripotency (Nichols et al., Cell 95:379-91, 1998).

Examples of polypeptides belonging to the Sox (SRY-box containing) family include, for example Sox1 (NM_009233, mouse and NM_005986, human), Sox3 (NM_009237, mouse and NM_005634, human), Sox7 (NM_011446, mouse and NM_031439, human), Sox15 (NM_009235, mouse and NM_006942, human), Sox17 (NM_011441, mouse and NM_022454, human) and Sox18 (NM_009236, mouse and NM_018419, human), and a preferred example includes Sox2 (NM_011443, mouse and NM_003106, human).

According to yet another embodiment, the method is effected by expressing in the cells four polypeptides—one belonging to the Oct family, one belonging to the Sox family, Nanog and LIN28.

According to yet another embodiment, the method is effected by expressing in the cells four polypeptides—one belonging to the Oct family, one belonging to the Sox family, KLF4 and LIN28.

Alternatively, the method is effected by expressing in the cells four polypeptides—one belonging to the Oct family, one belonging to the Sox family, KLF-4 and c-MYC.

Expressing the dedifferentiating factors described herein above in somatic cells may be performed by genetic manipulation—example using expression constructs. Various methods can be used to introduce the expression vectors of the present invention into the pancreatic beta cells. Such methods are generally described in, for instance: Sambrook, J. and Russell, D. W. (1989, 1992, 2001), Molecular Cloning: A Laboratory Manual, Cold Springs Harbor Laboratory, New York; Ausubel, R. M. et al., eds. (1994, 1989). Current Protocols in Molecular Biology, John Wiley and Sons, Baltimore, Md. (1989); Chang, P. L., ed. (1995). Somatic Gene Therapy, CRC Press, Boca Raton, Fla.; Vega, M. A. (1995). Gene Targeting, CRC Press, Boca Raton, Fla.; Rodriguez, R. L. and Denhardt, D. H. (1987). Vectors: A Survey of Molecular Cloning Vectors and Their Uses, Butterworth-Heinemann, Boston, Mass; and Gilboa, E. et al. (1986). Transfer and expression of cloned genes using retro-viral vectors. Biotechniques 4 (6), 504-512; and include, for example, stable or transient transfection, lipofection, electroporation, and infection with recombinant viral vectors. In addition, see U.S. Pat. Nos. 5,464,764 and 5,487,992 for positive-negative selection methods.

Introduction of the expression constructs of the present invention into somatic cells by viral infection offers several advantages over other methods such as lipofection and electroporation offering higher efficiency of transformation and propagation. According to a particular embodiment, expressing the dedifferentiating factors described herein above in the somatic cells is performed by retroviral transduction.

Methods of inducing iPS cells without viral integration are also contemplated—see for example Stadtfeld et al., 2008, [Science 322, 945-949] and Okita et al., 2008, [Science 322, 949-953].

Alternatively, somatic cells may be transfected with mRNAs encoding the dedifferentiating factors [Givol et al., BBRC 394 (2010): 189-193; Warren et al., Cell Stem Cell, Volume 7, Issue 5, 5 Nov. 2010, Pages 549-550] or by introduction of the proteins themselves (see for example Kim, D. et al. Cell Stem Cell doi: 10.1016/j.stem.2009.05.005 (2009) and Zhou, H. Et al. Cell Stem Cell 4, 381-384, (2009).

Examples of culture media which may be used whilst inducing and culturing iPS cells include DMEM, DMEM/F12, or DME culture solutions (these culture solutions may further appropriately contain serum (e.g. 10-15%), LIF, antibiotics, L-glutamine, nonessential amino acids, beta-mercaptoethanol, or the like) or commercially available culture solutions e.g., a culture solution for culturing mouse ES cells (TX-WES culture solution, Thromb-X).

Once cells of the cardiac organoid (e.g. cardiomyocytes, endothelial cells, fibroblasts, smooth muscle cells) are obtained they are cultured under conditions that promote formation of the multicellular organoid. Additional cells that may be added to the culture include pericytes and other supporting cells.

In one embodiment, the four cell types are mixed together prior to culturing in a suitable receptacle (e.g. a 24 well plate or a 96 well plate). In another embodiment, each cell type is added separately to the receptacle in a time-based manner.

In one embodiment, at least 50% of the cells used to make the organoid are cardiomyocytes, at least 60% of the cells used to make the organoid are cardiomyocytes or even at least 70% of the cells used to make the organoid are cardiomyocytes.

In another embodiment, the majority of cells used to make the organoid are cardiomyocytes and endothelial cells.

Exemplary ratios of cells used to engineer the organoid are provided in Table 1 of Example 2, herein below.

Exemplary conditions that promote formation of the multicellular organoid comprise insulin depravation and a culture period of at least 10 days.

The term “insulin depravation” refers to an amount of insulin which is below 5 μg/ml. In one embodiment, the amount of insulin is below 0.5 μg/ml. Thus, trace amounts of insulin may still be present in a culture medium. In a particular embodiment, the term “insulin depravation” refers to a basic culture medium to which no additional insulin is supplemented.

An exemplary method for promoting formation of the multicellular organoid includes:
(a) culturing cardiomyocytes, endothelial cells, fibroblasts and smooth muscle cells in a medium (e.g. RPMI-B27) being substantially devoid of insulin for at least two days, at least three days, or at least 5 days.
(b) supplementing the medium with VEGF to generate a supplemented medium; and
(c) culturing the cardiomyocytes, endothelial cells, fibroblasts and smooth muscle cells in the supplemented medium for at least 7 additional days, 10 additional days, 15 additional days. In one embodiment, the culturing does not exceed 30 days. In another embodiment, the culturing does not exceed 60 days.

Alternatively, the initial medium of step (a) may be removed and replaced by a new medium for step (b), the new medium being identical to the medium of step (a), except that it also includes VEGF.

Exemplary media that can be used for this process include for example Iscove's Dulbecco's Modified Eagle Medium (IMDM), RPMI. It will be appreciated that a person of skill in the art is capable of selecting appropriate media which supports the growth/differentiation of all the cell types in the organoid.

Optionally, the organoid is matured at least 10 days, 12 days, 15 days or 20 days, or even 30 days from initial establishment.

In one embodiment, the organoid is matured by culturing in a maturation medium comprising:
(i) an mTOR inhibitor; and
(ii) free fatty acids;

An mTOR inhibitor inhibits the function of mammalian target of rapamycin (mTOR). Exemplary mTOR inhibitors include, but are not limited to rapamycin, temsirolimus (CCI-779), everolimus (RAD001), and ridaforolimus (AP-23573). A commercially available mTOR inhibitor is Torin-1 manufactured by InvivoGen.

The mTOR inhibitor may be present in the medium at concentrations between 1 nM and 100 μM, between 1 nM and 10 μM, between 1 nM and 1 μM, between 1 nM and 100 nM.

Exemplary free fatty acids which may be used in the maturation medium include, but are not limited to linoleic acid, palmitic acid, propionic acid, arachidonic acid and linolenic acid.

The free fatty acid may be present in the medium at a concentration between 0.01 to 20%, 0.1-10% or 0.1-5%.

The maturation medium may further comprise additional components including but not limited to a steroid hormone (e.g. dexamethasone) and a thyroid hormone.

The amount of glucose in the maturation medium is typically between 1 mM-20 mM.

In one embodiment, the cardiac organoid is cultured in the maturation medium until the expression of TNNI3 (Troponin I, cardiac muscle) in the cardiomyoctes is at least 1.5 fold higher than the expression of TNNT2 (Troponin T2, cardiac type) in the cardiomyocytes, as measured by RT-PCR.

The matured cardiac organoid generated according to methods described herein has a structure which can be distinguished from human cardiac tissue retrieved from a mammalian organism (e.g. human). For example, the organoid is devoid of a layered organization of smooth muscle cells and/or fibroblasts—e.g. they are distributed evenly throughout the organoid.

Unlike a native cardiac organ, the organoid of the invention is devoid of a chamber.

Unlike a native cardiac organ, the organoid of the invention is devoid immune cells.

Unlike a native cardiac organ, the organoid of the invention is devoid of blood vessels greater than 10 μm in diameter.

In another embodiment, immune cells are supplemented to the organoid.

In one embodiment, the organoid is not printed (e.g. 3D-printing).

It will be appreciated that the maturation method described herein can be used for maturing any immature cardiac cell.

Thus, according to another aspect, there is provided a method of obtaining mature cardiomyocytes comprising:
(a) differentiating pluripotent stem cells into immature cardiomyocytes, wherein an abundance of TNNI1 or TNNI2 in an immature cardiomyocyte is at least 1.5 fold higher than an abundance of TNNT3 in said immature cardiomyocyte, as measured by Western Blot; and
(b) culturing said immature cardiomyocytes in a maturation medium comprising:
(i) an mTOR inhibitor; and
(ii) free fatty acids;
under conditions that promote maturation of the immature cardiomyocytes, wherein the protein abundance of TNNI3 is detectable and upregulated as measured by Western blot analysis.

Immature cardiomyocytes typically have a higher (e.g. at least 1.5 times, at least 2 times, at least 3 times at least 5 times) abundance of TNNI1 or TNNI2 as compared to TNNT3. An upregulation in the amount of TNNT3 above a predetermined amount (e.g. at least 2 fold, at least 3 fold, at least 4 fold, at least 5 fold (signifies that the cardiomyocyte has matured).

Methods of measuring the abundance of TNNI1, TNNI2 and/or TNNT3 are known in the art and may be carried out on the RNA or protein level. According to one embodiment, a Western blot is used to determine the amount of TNNI1, TNNI2 and/or TNNT3 protein.

The immature cardiomyoctes may be comprised in a single layered culture or a multi-layered culture.

The immature cardiomyocytes may be comprised in an organoid. The organoid can be multicellular (composed of at least 2, 3, 4, or more cell types), or unicellular (composed entirely of cardiomyocytes).

Heart failure with preserved ejection fraction (HFpEF) is a multifactorial disease associated with significant morbidity and mortality along with detrimental impacts on quality of life. HFpEF constitutes half of all heart failure cases, but unlike heart failure with reduced ejection fraction (HFrEF), there are almost no treatments for HFpEF that improve survival. One contributing factor to this issue may arise from the limited availability of in-vitro models for HFpEF, which hampers the advancement of new therapeutic approaches. There are currently no models based on human tissues which could further impede the identification of relevant novel therapeutic targets. An additional complexity in the modeling of HFpEF arises from the multifactorial nature of the disease, commencing from the confluence of various comorbidities, including but not limited to obesity, hypertension, and diabetes.

The present inventors have now generated a novel human in-vitro platform that emulates the typical and most common pathological conditions associated with HFpEF. They have shown that exposure of the organoids to a combination of comorbidity-inspired conditions resulted in a synergistic effect ultimately manifesting as hypertrophy, ultrastructural fibrosis and abnormal relaxation, the pathophysiological hallmarks of HFpEF. Consequently, the present inventors opted to utilize the combination of the comorbidities-mimicking conditions as the HFpEF inducers. Through comprehensive physiological assessments that integrate dynamics of force-length relationships, they illustrated that subjecting the organoids to the HFpEF inducing conditions leads to significantly increased passive stiffness, extended relaxation periods, and prolonged calcium decay. Finally, the findings reveal that cardiac organoids exposed to HFpEF inducing conditions exhibit the key pathophysiological characteristics of the disorder such as increased levels of reactive oxygen species and compromised energetic status as evidenced by reduced oxygen consumption rates and diminished ATP content.

Thus, according to another aspect of the invention there is provided a method of generating a cell model for Heart failure with preserved ejection fraction (HFpEF) comprising:
culturing human cardiomyocytes in a medium comprising at least one hypertension-associated hormone and at least one metabolic disease promoting agent, under conditions that bring about at least one, at least two, at least three or all of the following phenotypes:
   (i) an increase in expression of collagen in the cardiomyocytes as compared to prior to the culturing;
   (ii) an increase in relaxation time of the cardiomyocytes (e.g. by at least 20%, 25%, 30%, or more) as compared to prior to the culturing;
   (iii) an increase in production of NT-proBNP by the cardiomyocytes as compared to prior to the culturing; or
   (iv) an increase in cardiomyocyte size (e.g. by at least 20%, 25%, 30%, or more) as compared to prior to the culturing.

The human cardiomyocytes of this aspect of the invention may be from a human cardiac cell line i.e. immortalized (for example HL-1 cell line or AC16 cell line).

Alternatively, the human cardiomyocytes may be primary cells derived from a biopsy of a subject.

Still alternatively, the human cardiomyocytes may be ex vivo differentiated from pluripotent stem cells including embryonic stem cells, mesenchymal stem cells, induced pluripotent stem cells etc., as described herein above.

The human cardiomyocytes may be mature cardiomyocytes or immature cardiomyocytes. Methods of maturing human cardiomyocytes are described herein above.

In one embodiment, the human cardiomyocytes are comprised in an organoid, including multicellular organoids (such as those described herein above) or unicellular organoids (composed of cardiomyocytes). Other methods of generating cardiac organoids are described in Example 3, herein below,

Expression of collagen may be measured on the RNA or protein level using methods know in the art. According to a particular embodiment, collagen expression is increased by at least 1.5 fold, at least 2 fold, at least 3 fold, at least 5 fold.

Expression of NT-proBNP may be measured on the RNA or protein level using methods know in the art. According to a particular embodiment, collagen expression is increased by at least 1.5 fold, at least 2 fold, at least 3 fold, at least 5 fold.

Measurement of cardiomyocyte size may be carried out my microscopy or other methods known in the art. For example, hypertrophy can be measured via: tissue size analysis using bright-field (BF) microscopy, immunofluorescence staining for intracellular proteins/structural proteins/membrane-binding dyes followed by computational analysis.

Examples of hypertension-associated hormones include, but are not limited to Angiotensin-II and/or Endothelin-1.

Metabolic disease promoting agents include obesity promoting agents (e.g. fatty acids and inflammatory cytokines) and diabetes promoting agents (e.g. an amount of glucose above 10 mM).

According to a particular embodiment, the human cardiomyocytes are cultured in a medium comprising:
Angiotensin-II and/or Endothelin-1;
fatty acids,
IL-1β,
IFN-γ
glucose at a concentration above about 10 mM,
The medium is preferably devoid of insulin.

The cell model obtained according to the methods described herein may be used in basic scientific research of HFpEF.

The cell model obtained according to the methods described herein above is useful for determining whether an active agent is useful for treating HFpEF.

Candidate agents are contacted with the HFpEF model described herein. The candidate agent may be a small molecule, a protein, a nucleic acid, a hormone, a known drug, an antibody.

After a predetermined amount of time (e.g. 1 hour, 6 hours, 12 hours 24 hours), the cardiomyocytes of the model are analyzed. At least one of the following characteristics of the cardiomyocytes is analyzed:
(i) expression of collagen;
(ii) relaxation time;
(iii) production of NT-proBNP;

A decrease in expression of collagen in the cardiomyocytes by a predetermined amount, a decrease in relaxation time of the cardiomyocytes by a predetermined amount, and/or a decrease in production of NT-proBNP by a predetermined amount as compared to prior to the contacting is indicative of an agent useful for treating HFpEF.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Generation of Human Cardiac Organoids

### Materials and Methods

hiPSC generation and culture: Dermal fibroblasts were obtained from healthy individuals. Fibroblasts were reprogrammed to generate the patient-specific human induced pluripotent stem cells (hiPSCs) clones by retroviral delivery of three reprogramming factors as previously described (SOX2, KLF4, and OCT4) (Takahashi, K. & Yamanaka, S. Cell 126, 663-676 (2006)

Undifferentiated hiPSCs were grown in mTeSR Plus (Stemcell Technologies, Vancouver, Canada) on growth-factor-reduced Culturex® (R&D, US)-coated 6-well plates and passaged every 4 days at 1:10 ratio using 5 mM EDTA solution (Life Technologies, California, USA) and 2 μM ROCK inhibitor Thiazovivin (Cayman, US).

Differentiation of hiPSCs into cardiomyocytes (CMs): The method was based on a well-established cardiomyocyte differentiation protocol (Lian, X. et al. Proc Natl Acad Sci USA 109, E1848-57 (2012)). In short, hiPSCs were differentiated into cardiomyocytes by applying CDM3 media with 6 μM CHIR99021 (Tocris, Bristol, UK) with cell confluency of 85%. After 2 days, the medium was changed to CDM3 with 2 μM Wnt-C59 (Selleck Chemicals, USA). 2 days later, the medium was changed into RPMI-1640 (Gibco Dynamics Medium, Thermo Fisher, Massachusetts, USA) with B27 supplement (Thermo fisher, Massachusetts, USA) without insulin.

Differentiation of hiPSCs into Endothelial cells (ECs): hiPSCs were dissociated using EDTA and plated on a 24-well plate with growth-factor-reduced Culturex® (R&D) at a density of 50,000 cells cm-2 in mTeSR with 2 μM ROCK inhibitor Thiazovivin. After 72 h, the medium was replaced with LasR basal medium (Advanced DMEM: F12 with Glutamax and Ascorbic acid) with 6 μM CHIR to induce the cell to mesodermal fate. After 2 days, the same medium was used but now supplemented with 200 ng/ml VEGF to induce the mesodermal cells into endothelial progenitor cells (induction media). The above-mentioned induction media was renewed day after day, for the next three days. On day six of the differentiation, cells were dissociated using Accutase (STEMCELL Technologies, Canada), bound with CD31⁺ magnetic beads, then sorted using MACS sorting.

Following sorting, pure endothelial cells were cultured in a StemPro-34 medium supplemented with 50 ng/ml VEGF.

Differentiation of hiPSCs into epicardial cells: hiPSCs were dissociated using EDTA and plated on a 12-well plate with growth-factor-reduced Culturex® (R&D, US) at a density of 50,000 cells cm-2 in mTeSR with 2 μM ROCK inhibitor Thiazovivin. After 48 h, on day 0, media was replaced with RPMI Basal medium supplemented with B27-insulin supplement and 6 μM CHIR99021 (a GSK-3B inhibitor will be applied for 48 hours to initiate mesodermal induction). After 48 h, media was replaced with the same RPMI medium with 2.5 μM IWP2, and on day 4 of the differentiation the medium was refreshed without any supplementation. At this stage, the cells are considered cardiac progenitors. From day 6, cells were replated as single cells at a density of 40,000 cells per cm² on gelatinized 12-well plates in a 5 μM ROCK inhibitor Thiazovivin-containing LaSR basal medium. The medium included Advanced DMEM: F12, 2.5 mM GlutaMAX, and 100 μg/mL ascorbic acid. On days 7 and 8, media were changed daily with LaSR basal medium supplemented with 3 μM CHIR. The cobblestone-like epicardial cell morphology was detected as early as day 8. From days 9 to 11, cells will be incubated with fresh LaSR medium every day.

Cardiac Fibroblasts Generation—Epithelial-Mesenchymal Transition (EMT): Epicardial cells were induced to differentiate into fibroblasts by supplementing the LaSR basal medium with 10 ng/ml bFGF for 6 following days. bFGF-treated cells adopt a fibroid spindle-like morphology typical of cultured fibroblasts, whereas non-treated cells preserve a cobblestone-like appearance typical of Epithelial cells.

Smooth Muscle cell generation—Epithelial-Mesenchymal Transition (EMT): epicardial cells were induced to differentiate into smooth muscle cells by supplementing the LaSR basal medium with 10 ng/ml bFGF and 5 ng/ml TGFB (TGF beta) for 6 following days. Smooth muscle cells can be identified by FACS and immunostaining for smooth muscle actin.

Micro-Organoid formation: hiPSC-derived cardiomyocytes grown as monolayers were detached using TrypLE express X1 (Thermo Scientific, Massachusetts, USA) for 5 minutes at 37° C. hiPSC-derived endothelial cells, cardiac fibroblasts, and smooth muscle cells were also grown as monolayer and detached using Accutase (STEMCELL Technologies, Canada) for 1-2 minutes at 37° C. The detached cells were seeded on a V-bottom 96 well plate (Thermo Fisher, Massachusetts, USA) in a concentration of 10⁴ cells/50 μL for each well. Microplates were then centrifuged for 10 min at 1100 rpm. Micro-organoids were incubated at 37° C., 5% CO₂ for 3 days with RPMI-B27 media without insulin and in the presence of vascular endothelial growth factor (VEGF), and cultured for 16 days.

Maturation of micro-organoid: as described in Example 2. Glucose concentration was 11.1 mM.

Contraction analysis: After treatment, video recordings were carried out using a robotic inverted microscope (Olympus IX83, Olympus, USA). The organoids were video recorded for 8 seconds (for each well) in an Okolab Incubator (Italy) at the temperature of 37° C. and with 5% CO₂. The videos were analyzed using the MUSCLEMOTION tool for ImageJ (Freeware). All signals shorter than 200 ms and longer than 600 ms were excluded. Outliers were calculated using a Python script that calculates the quantiles, IQR, and upper and lower limits. The outlier signals were excluded from the calculation of the average of the micro-organoid signal. Bazett formula was used for calculating the contraction duration and relaxation time.

Gene expression (RT-qPCR): RNA was extracted from cells using CellTiter-Glo 2.0 Assay (Promega, US), then reverse transcribed to cDNA with the Iscript™ cDNA Synthesis Kit (Bio-Rad, US). The quantitative polymerase chain reaction was performed using the Lightcycler® 480 SYBR Green I Master (syber green) (Roche, Switzerland).

Western blot: Cells were lysed with lysis buffer RIPA with 1:1000 0.5M DTT and 1:100 protease inhibitor. The cell lysate was sonicated (5-sec sonication and 30-sec breaks for four cycles) and centrifuged for 10 min at 4° C. Samples were loaded into 15% gel and electrophoresed for 1 hour at 120 V before transfer to a PVDF membrane using the Trans-Blot Turbo Transfer System (Bio-Rad, US).

The SuperMarker2700 3-color Pre-Stained Protein Marker ladder was used to estimate the molecular weight of detected bands (Bio-Lab). Membranes were blocked with 1% milk or bovine serum albumin and then incubated with the primary antibody mab1691 (1:1000) antigen for troponin I overnight at 4° C. followed by incubation with secondary antibody-Goat anti-mouse IgG H&L (1:5000) (IRDye 800CW) for 1 hour at room temperature.

ATP test: Cells were detached using TrypLE express X1 (Thermo Scientific, Massachusetts, USA) for 5 minutes at 37° C. (for CMs) or Accutase (STEMCELL Technologies, Canada) for 1-2 minutes (for ECs, CFs, and SMCs) and seeded on a 24-well optical plate covered with growth-factor-reduced Culturex® (R&D, US) or 0.1% gelatin. Cells were exposed to maturation media and control media for a week. On the day of the measurement, the plate was equilibrated to room temperature for approximately 30 minutes. 1 ml of tested media mixed with 1 ml of CellTiter-Glo® 2.0 Reagent (Promega, US) was added to each well and the plate was mixed for 2 minutes on an orbital shaker, then incubated at room temperature for 10 minutes.

The plate was scanned with a plate reader infinite m plex (Tecan, Switzerland).

Tube formation: ECs were exposed to maturation media and control media for a week. 48-well plate was covered with 150 μl per well of growth-factor-reduced Culturex® (R&D, US) and incubated for 1 hr at 37° C. 35K cells were plated for 24 hours. Tube formation is quantified using the live-content imaging system-IncuCyte. The analytical evaluation of these assays was performed using “Angiogenesis Analyzer” for ImageJ.

Immunofluorescence analysis—Micro-Organoids: Micro-organoids were treated with maturation media, as described in Example 2, for 14 days, then fixated for 30 min with 20% triton and 35% formaldehyde in 1.5 ml Eppendorf. Micro-organoids were washed 3 times with PTX (PBS+0.1% triton) and blocked for 1 hr at room temperature with 1 ml blocking buffer (PTX+0.2 gr BSA+200 μl DHS) and stained with primary antibodies (1:250 cTnT ab91605 and 1:250 CD31 ab9498, Abcam, UK) diluted in blocking buffer overnight at 4° C. The day after Micro-organoids were washed in PTX and stained for 1 hr with secondary antibodies (1:300 Alexa Fluor 594 and 1:300 Alexa Fluor 488) diluted in blocking buffer and DAPI. Micro-organoids were washed with PTX and PBSx1 and mounting media was added for 1 hr. Micro-organoids were scanned with confocal Laser Scanning Microscopy (Zeiss 900).

### Results

Overall morphology and cellular architecture in both matured and non-matured (control) multi-cellular micro-organoids was examined by immunofluorescence (staining for cardiomyocytes, with α-actinin) and endothelial cells with α-CD31) (FIG. 1A). Multicellular micro-organoids started to develop vessel-like structures which demonstrates the functional interplay between the endothelial cells and the other supporting cells. In addition, the H&E stain also revealed vessel-like structures in the matured multicellular micro-organoids (FIG. 1B). RT-qPCR confirmed the presence of ECs and SMCs using specific marker genes-CADH5 and SMTN respectively in the matured organoids (FIG. 1C).

### Example 2

### Maturation of Cardiac Cells and Cardiac Supporting Cells

### Materials and Methods

Micro-organoid formation: generated as described in Example 1.

Two compositions of multicellular Micro-Organoids were tested; the proportions are indicated in Table 1, herein below.

The first composition has a high proportion of cardiomyocytes and a low level of supporting cells. The second has a higher proportion of supporting cells.

TABLE 1

| | High CMs | Low CMs |
|---|---|---|
| CMs | 65% | 50% |
| ECs | 25% | 35% |
| CFs | 5% | 10% |
| SMCs | 5% | 5% |

Cardiomyocyte maturation: hiPSCs were differentiated into cardiomyocytes as follows. On day 0, the hiPSC medium was replaced with CDM3 media supplemented with 6 μM CHIR99021 (R&D Systems, US) to induce differentiation toward a mesodermal fate. After 48 hours the same medium was use, supplemented with 2 μM of Wnt-C59 (Selleck Chemicals, US). After 48 hours, the medium was replaced with RPMI-B27-insulin (Thermo Fisher Scientific, US). On day 10, maturation process was started with the replacement of the medium with maturation media. This process continued for 14 days during which Torin1 was added only in the first 7 days. At the end of the process, several criteria were tested to characterize the maturation of the CMs and the other supporting cells. The maturation media (DMEM) comprised the following components listed in Table 2.

TABLE 2

| Component | Concentration |
|---|---|
| DMEM media | |
| 11-966-025 | |
| Fisher Scientific | |
| vitamins | B12 - 5 μg/ml |
| Vitamin B12 Sigma Aldrich, V6629 | Biotin - 0.82 μM |
| Biotin Sigma Aldrich, B4639 | |
| Free Fatty Acids (Albumax) | 0.5% |
| Rhenium 11020021 | |
| Low glucose/high glucose | 3 mM/11.1 mM |
| Sigma-Aldrich 122100000592112 | |
| Lactate | 10 mM |
| Sigma-Aldrich 71718 | |
| B27 supplement -insulin | |
| Rhenium17504044 | |
| Knockout serum replacement | 1% |
| Fisher Scientific10-828-028 | |
| Adult cardiomyocyte media components | L-carnitine - 2 mM |
| (L-carnitine C3630 Sigma-Aldrich, taurine | Taurine - 2 mM |
| T0625 Sigma-Aldrich, | Creatine - 5 mM |
| creatine C3630 Sigma-Aldrich, | Ascorbic acid - 0.5 mM |
| ascorbic acid A8960 Sigma-Aldrich) | |
| Hormones (dexamethasone 230300010 Acros | Dexamethasone - 100 ng/ml |
| Organics), | T3 - 74.25 nM |
| thyroid hormones T0281 Merk) | |
| mTOR inhibitor (torin-1) | 10 nM |
| R&D Systems 4247/10 | |

Effect of Torin1 on maturation: cardiomyocytes were cultured in the following media the following media in the presence of two concentrations of Torin1 (10 nM and 25 nM):
RPMI-B27+Torin1 10 nM.
RPMI-B27+Torin1 25 nM.
Maturation media with 3 mM glucose.
Maturation media+Torin1 10 nM.
Maturation media+Torin1 25 nM.
Control-RPMI-B27.

### Results

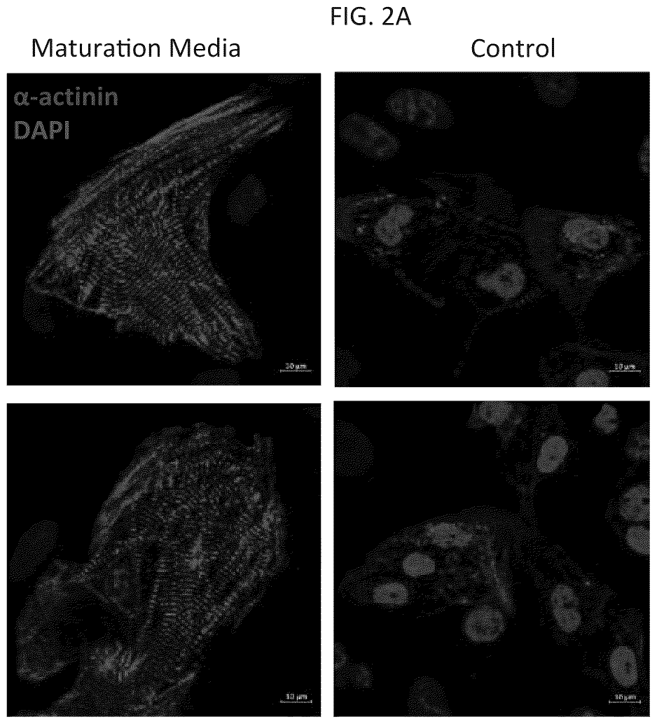

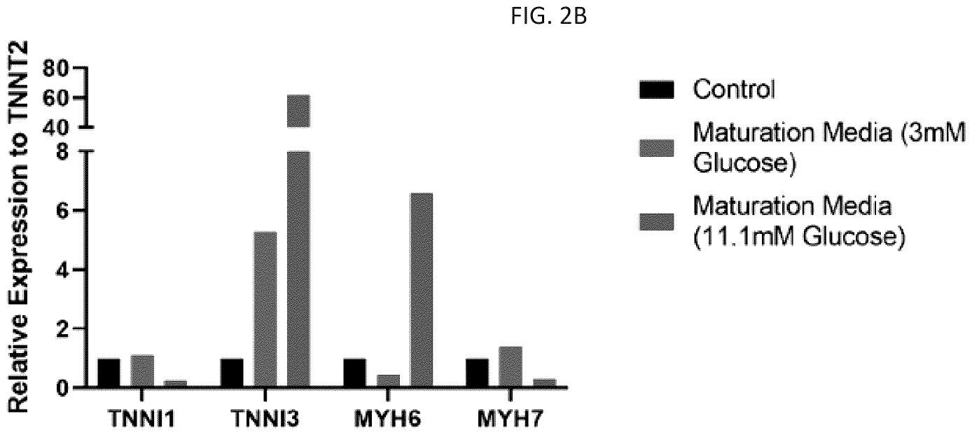

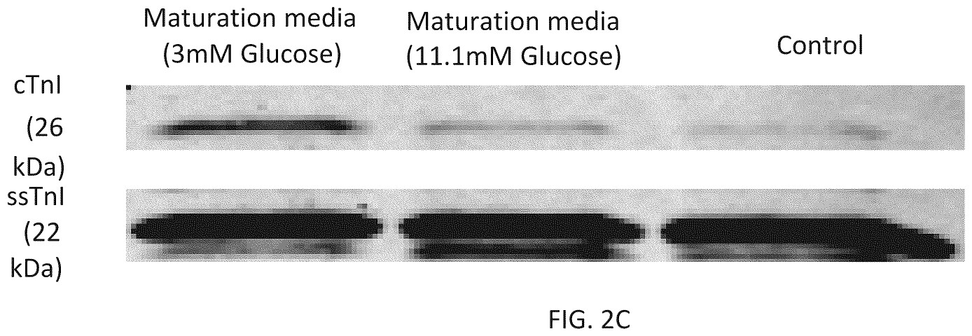

Immunostaining studies of CMs following treatment with 11.1 mM glucose-maturation media revealed a high degree of sarcomere maturation with longer sarcomere length and anisotropic-like orientation (FIG. 2A).

Real-time PCR demonstrated a significant increase in TNNI3 expression in both low and high glucose maturation media groups. Western blot analysis demonstrated that the exposure to 3 mM of glucose resulted in a higher degree of cTnI (FIG. 2C).

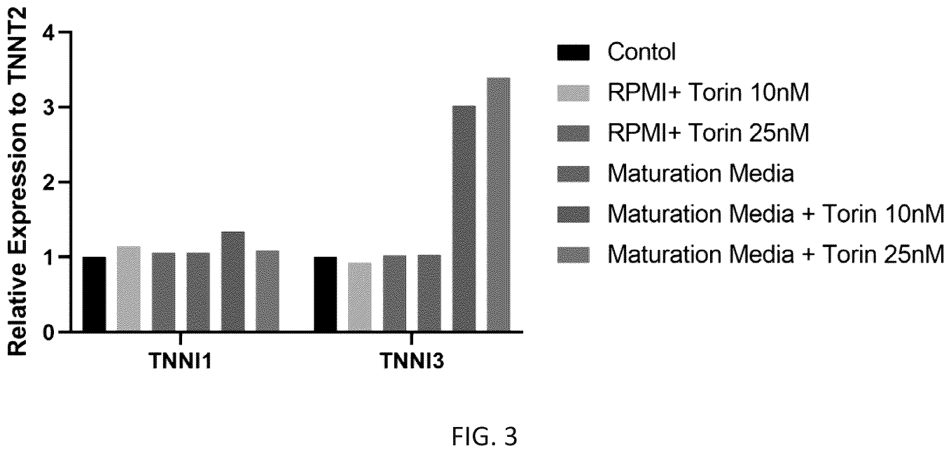

As illustrated in FIG. 3, Torin1 treatment trended toward increased expression of TNNI3 mRNA expression in a dose-dependent manner in the maturation media groups.

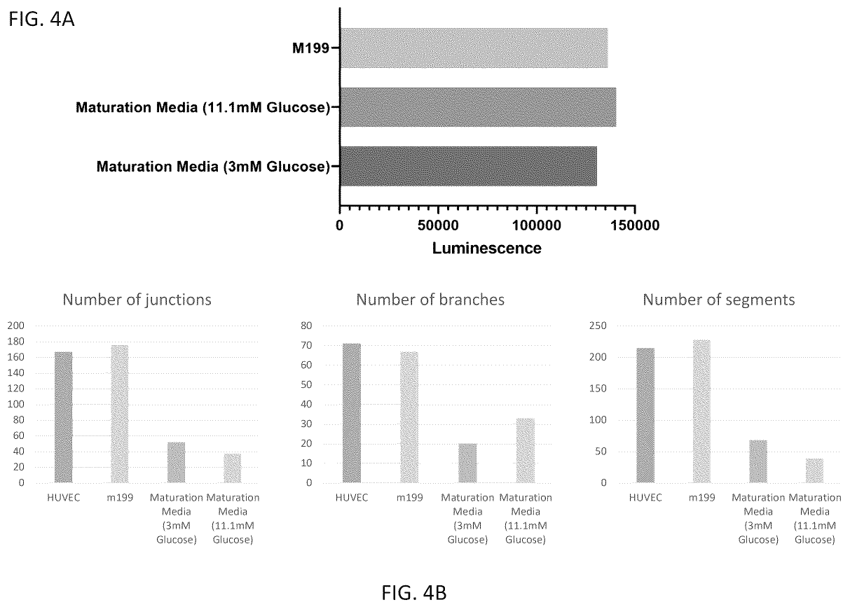

Next, the viability of hIPS cell-derived endothelial cells in maturation media (3 mM glucose and 11.1 mM glucose) was compared with the viability in control media (m199). As demonstrated in FIG. 4A, the highest luminescence was obtained in the maturation media in the 11.1 mM glucose group.

Next, a tube formation assay was performed comparing between endothelial cells (hiPSC-ECs and HUVECs) treated with maturation media (3 mM glucose and 11.1 mM glucose). As demonstrated in FIG. 4B, hiPSC-ECs treated with maturation media (3 mM glucose and 11.1 mM glucose) created junctions (52 and 37), branches (20 and 33), and segments (68 and 39). HUVECs and hiPSC-ECs treated with control media created a higher number of these structures (junctions (167 and 176), branches (71 and 67), and segments (215 and 228)).

Maturation Media Promote Structural and Functional Maturation of hiPSC-CMs in Micro-Organoids:

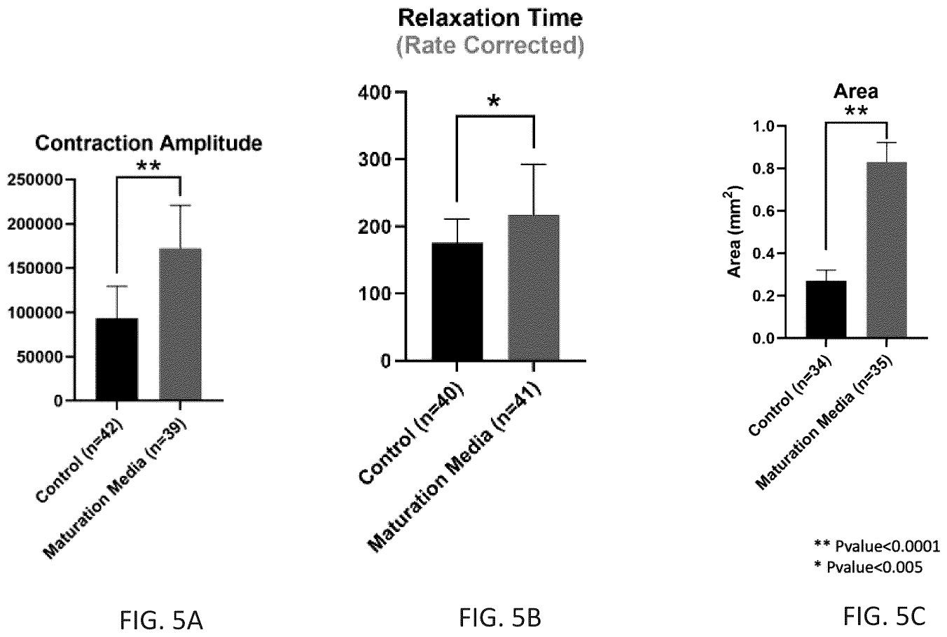

A unicellular micro-organoid with only cardiomyocytes was initially tested. When using a unicellular model, the contraction amplitude was significantly improved when the maturation process was applied, 93,489.85±35,993.07 a.u for the control group versus 172,181.38±48,5004.5 a.u for the maturation group (arbitrary units of contraction amplitude) (p<0.0001) (FIG. 5A). Relaxation dynamics also improved (FIG. 5B). The area of the mature micro-organoids (0.82±0.09 mm²) was significantly increased compared to the control group (0.27±0.05 mm²) (p<0.0001) (FIG. 5C).

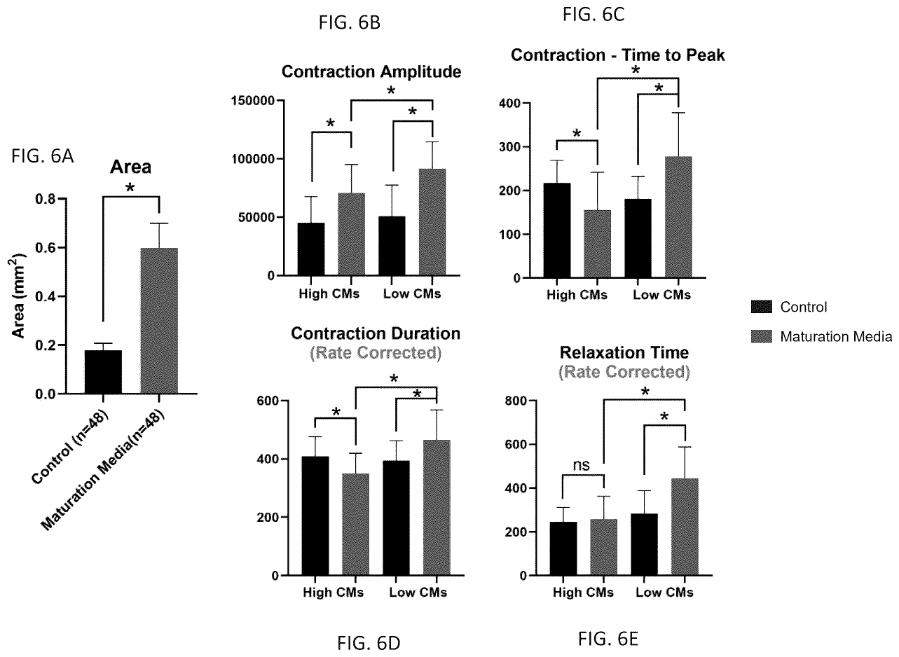

Next, the multicellular micro-organoid was tested. A significant increase in the area of the matured multicellular organoids was observed (0.6±0.1 mm² versus 0.18±0.03 mm²) (p<0.0001) (FIG. 6A). Immunostaining study revealed that the multicellular organoids in both the control and the maturation groups started to develop vessel-like structures (FIG. 1A) which demonstrate the functional interplay between the endothelial cells and the other supporting cells.

The maturation process was next tested on the high cardiomyocyte microorganoid and the low cardiomyocyte organoid. Both cellular compositions matured to a level that resulted in higher force generation compared to the control (in the high CMs group 44,971.33±22,617.34 a.u for the control group versus 70,544.84±24,639.18 a.u for the maturation group and the low CMs group 50,745.59±26,647.93 a.u for the control group versus 91,553.31±22,850.62 a.u for the maturation group, p<0.0001) (FIG. 6B). The low cardiomyocyte composition generated a significantly higher contraction amplitude. However, contraction dynamics were improved along the maturation process in the high cardiomyocyte group (the contraction duration of the control group was 408.94±67.98 ms versus 349.74±69.92 ms in the maturation group, the contraction time to peak of the control group was 217.26±51.9 ms versus 155.49±85.94 ms in the maturation group, p<0.0001), while these were prolonged for the low cardiomyocyte group (the contraction duration of the control group was 394.31±68.64 ms versus 465.81±102.28 ms in the maturation group, the contraction time to peak of the control group was 181.03±51.21 ms versus 277.83±100.44 ms in the maturation group, p<0.0001) (FIGS. 6C-E).

### Example 3

### Generation of HFpEF Model

### Materials and Methods

hiPSC generation and culture: Dermal fibroblasts were obtained from healthy individuals. Fibroblasts were reprogrammed to generate the patient-specific human induced pluripotent stem cells (hiPSCs) clones by retroviral delivery of three reprogramming factors as previously described (SOX2, KLF4, and OCT4) (Takahashi, K. & Yamanaka, S. Cell 126, 663-676 (2006).

Undifferentiated hiPSCs were grown in mTeSR Plus (Stemcell Technologies, Vancouver, Canada) on growth-factor-reduced Culturex® (R&D, US)-coated 6-well plates and passaged every 4 days at 1:10 ratio using 5 mM EDTA solution (Life Technologies, California, USA) and 2 μM ROCK inhibitor Thiazovivin (Cayman, US).

Human IPSCs-derived cardiomyocyte differentiation: In short, hiPSCs were differentiated into cardiomyocytes by applying CDM3 media with 6 μM CHIR99021 (Tocris, Bristol, UK) with cell confluency of 85%. After 2 days, the medium was changed to CDM3 with 2 μM Wnt-C59 (Selleck Chemicals, USA). 2 days later, the medium was changed into RPMI-1640 (Gibco Dynamics Medium, Thermo Fisher, Massachusetts, USA) with B27 supplement (Thermo fisher, Massachusetts, USA) without insulin. The medium was refreshed on days 7 and 9. On day 10 and forth, the medium was replaced with CDM3 (RPMI 1640 (Gibco, USA), 500 μg/ml recombinant human albumin (Sigma-Aldrich), 213 μg/ml L-ascorbic acid 2-phosphatase (Burridge et al)) and refreshed every two days.

Micro-organoids construction-hIPSCs-derived cardiomyocytes were detached using TrypLE express X 1 (Thermo Fischer, USA) for 5 minutes at 37° C. The detached cells were seeded on a V-bottom 96 well plate (Thermo Fisher, USA) in a concentration of 10⁴ cells/50 μl for each well. Microplates were then centrifuged for 10 min at 1100 rpm. Micro-organoids were incubated at 37° C., 5% CO₂ for 3 days with RPMI-B27 media without insulin. The medium was refreshed every 48 hours.

Cardiac organoids media—The media was based on DMEM medium without glucose (Thermo Fischer, USA) with added 3 mM glucose (Merk, USA) supplemented with B27 minus insulin (Thermo-Fisher Scientific, USA). 0.5% free fatty acids (FFAs) in the form of Albumax (Thermo Fischer Scientific, USA) 10 mM lactate (Merk, USA), 5 μg/ml B12-vitamin, 0.82 μM biotin, 5 mM creatine monohydrate, 2 mM taurine, 2 mM L-carnitine, 0.5 mM ascorbic acid (all from Merk, USA), 1% knockout serum KOSR, and 1% non-essential amino acids, 100 ng/ml dexamethasone, 4 nM Triiodo-L-thyronine (all from Thermo Fischer Scientific, USA), and 1% penicillin/streptomycin (Biological Industries Beit-Haemek, Israel).

Micro-organoids optical characterization—Organoids were monitored using an automated imaging system. ORCA-Flash4.0 camera (Hamamatsu, Japan) was mounted on an Olympus IX83 microscope with 4×/0.16 objective (Olympus, Japan). Organoids were automatically scanned using the TANGO Desktop stage controller (Märzhäuser Wetzlar, Germany). Organoids' size was analyzed using Cell Profiler software (Broad Institute) and Micro-organoid function (contraction and relaxation properties) were analyzed using Musclemotio, a FIJI software plugin.

Fibrosis Analysis—Micro-organoids were grown in HFpEF-inducing conditions and isolated comorbidities mimicking conditions. Organoids were then fixed using 4% paraformaldehyde followed by Masson-Trichrome staining. Imaging was conducted using panoramic 250 Flash III automatic slide scanner 250 (3DHISTECH, Hungary). Collagen content was analyzed using Cell Profiler software (Broad Institute).

Human cardiac organoid (HCO) construction: 2 million cIPSC-derived cardiomyocytes were dissociated and mixed with bovine collagen (LLC Collagen solutions, USA), 2×DMEM (Gibco, USA) and 0.1 M NaOH for pH neutralization. The mix was cast in ring-shaped mold and held for 3 days for condensation and transferred to a passive stretching device. HCO medium was refreshed every 2 days.

2×DMEM-40% 5×DMEM (Biological industries, Israel), 40% FETAL BOVINE SERUM (Biological industries, Israel), 15% H₂O, 10 μl/ml glutamine, and 20 μl/ml penicillin/streptomycin.

HCO Medium-Iscove-Medium with 20% fetal bovine serum, 1% nonessential amino acids, 1% glutamine, and 100 μmol/l β-mercaptoethanol.

Force-length relationship evaluation: HCOs were measured using a force transducer with a length controller (Aurora-Scientific, Canada). The organoids were paced in 1 Hz and stretched in 0.1 mm every 10 seconds up to 0.8 mm.

HFpEF-induction: Cardiomyocytes in 2D cultures, micro-organoids and the passively stretched HCOs were all cultured in basal cardiac organoids medium with added factors emulating the comorbidities associated with HFpEF: obesity-related inflammation, diabetes, and hypertension, and were referred to as “HFpEF-inducing conditions”. To emulate obesity-related inflammation, free fatty acids were added to the medium in the form of 0.5% concentration Albumax (from Thermo Fisher Scientific, USA) and inflammatory cytokines 10 ng/ml IL-1ß and 100 ng/ml IFN-γ (from Thermo Fisher Scientific, USA). Diabetic conditions were modeled by using high glucose concentration (11.1 mM) with insulin deprivation. Finally, hypertension was emulated by using 10 ng/ml Angiotensin-II and 10 ng/ml Endothelin-1 (both from Merck, USA). All cells and organoids were exposed to the HFpEF-inducing conditions for 7 days. The cells, tissues, and organoids were cultured in physiological conditions: 5% CO₂ at 37° C.

Calcium imaging: Cardiomyocytes were dissociated and seeded on 35 mm Glass bottom dish with 10 mm micro-well #1.5 cover glass (from Cellvis, USA) as single cells (50,000 cells/ml). Calcium imaging was performed using Fluo-4 calcium dye (from Thermo Fisher Scientific, USA) in a confocal microscopy platform Guatimosim et al. 2011).

NT-proBNP measurement: NT-proBNP was measured using Abbott Laboratories NT-proBNP kit from the 1:20 diluted media of the passively stretched organoids.

ATP Content—Cells were seeded on a 96 optic plate (Thermo Fisher Scientific, USA), and analyzed for their ATP content using CellTiter-Glo 2.0 Assay kit (Promega, USA) following manufacture instructions. The ATP content was assessed via Infinite M200 PRO plate reader (Tecan Trading AG, Switzerland).

Sea-Horse Metabolic Assay Test—Cardiomyocytes were seeded at 50 k cells/well density in Seahorse XF cell culture plates (By Agilent, USA), ensuring even distribution across the wells. The cells were exposed to HFpEF-inducing conditions and to cardiac organoids media as a control. The instrument automatically records the oxygen consumption rate (OCR) as indicators of mitochondrial respiration. The OCR was analyzed using Wave 2.6.3 (By Agilent, USA).

ROS measurement—Cardiomyocytes were seeded and allowed to attach in a 96-well plate and analyzed for their ROS content via DCFDA/H2DCFDA-Cellular ROS Assay Kit (abcam, Britain). The quantification of fluorescence intensity indicative of ROS levels was performed using Infinite M200 PRO plate reader (Tecan Trading AG, Switzerland).

Quantitative real-time PCR—For qPCR, total RNA was purified with Bio-Tri Reagent (BioLabs, Israel) following manufacture extraction protocol. RNA (1 μg) underwent reverse transcription using Iscript cDNA Synthesis Kit (Bio-Rad, USA). Real-time qPCR was performed using LightCycler 480 SYBR Green I Master (Roche, Switzerland). StepOnePlus Real-Time PCR System (Thermo Fisher Scientific, USA) was used to assess gene expression levels. Results were analyzed using comparative threshold cycle (Ct) method. Analysis employed the comparative Ct method, generating ΔCt values by subtracting the Ct of the house-keeping gene GAPDH Ct from each mRNA Ct in every sample. Relative expression was calculated using 2\hat{ }(ΔΔCt).

Statistical Analysis—Shapiro-Wilk test was used to assess whether the data are distributed normally. Normally distributed data sets are presented as mean±standard error measurement (SEM). Non-normally distributed data are presented as median and interquartile range (IQR). Comparisons among groups were analyzed with two-tailed student's t-test or ANOVA for normally distributed data and Mann-Whitney U test and Kruskal-Wallis for non-normally distributed data. Whenever multiple comparisons were performed, post-Hoc Tukey's test was used. For the evaluation of force-length relationships in hHCOs, Pearson's correlation coefficient was calculated, and a linear regression model was used. P value <0.05 was considered significant. The statistical analysis was performed using GraphPad Prism 9.5.1.

Fibrosis Analysis: Micro-organoids were stained using Masson-trichome and collagen content was analyzed using Cell Profiler software from Broad Institute (Stirling et al. 2021).

### Results

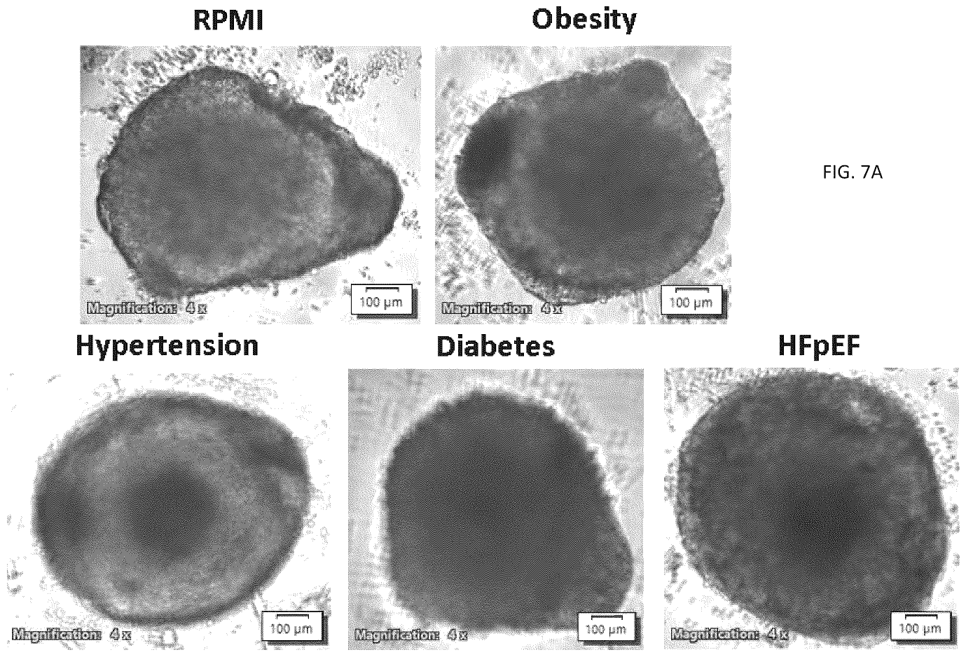

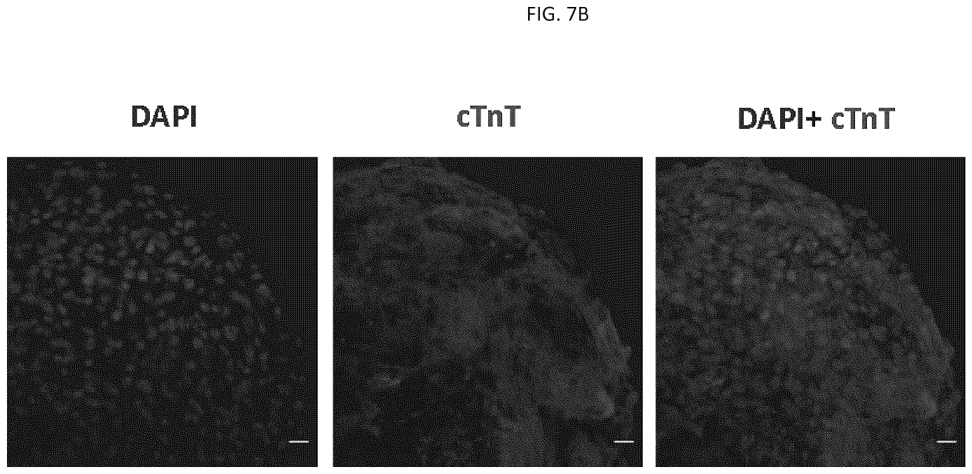

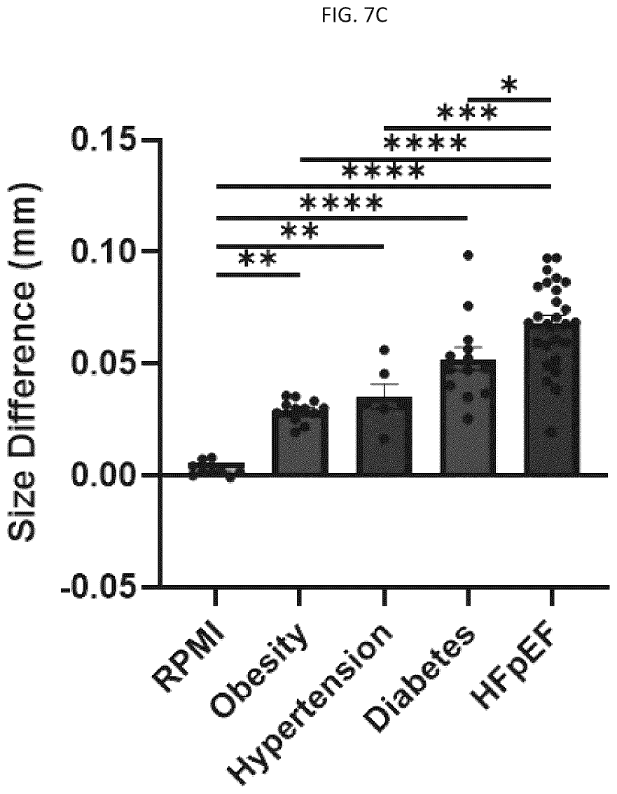

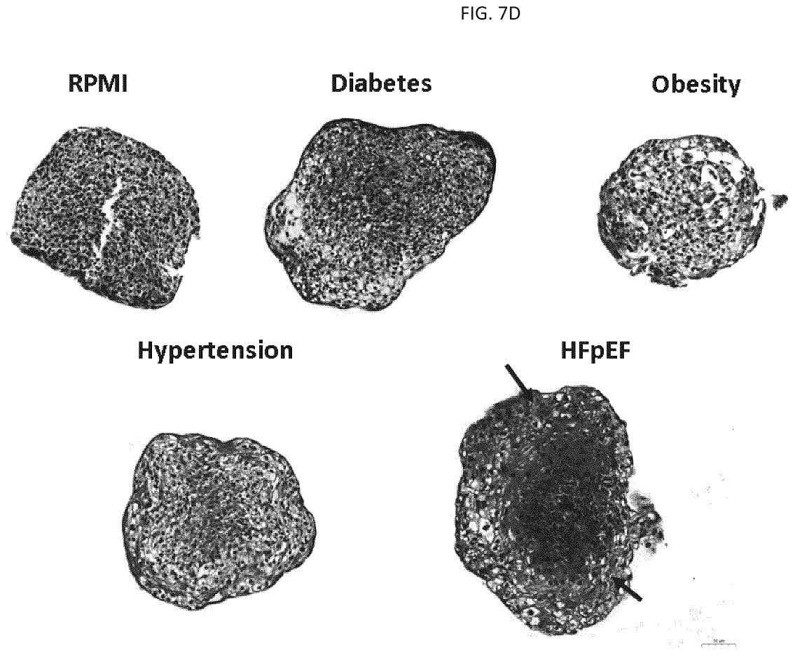

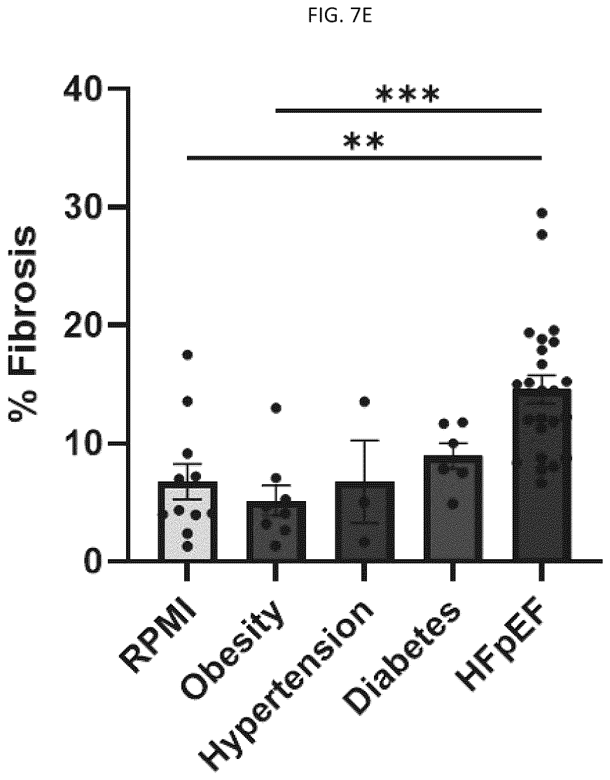

To evaluate the contribution of the conditions mimicking HFpEF-related comorbidities on HFpEF induction, a high throughput micro-organoids (10⁴ cells per organoid) screening platform was used. The micro-organoids were exposed to comorbidity-inspired inducing conditions commonly associated with HFpEF: hypertension, obesity-related inflammation, and diabetes mellitus. The effect of each isolated condition was evaluated and compared to the control medium, RPMI-B27, and to the combination of all conditions associated with HFpEF. Organoid morphology on bright field imaging is depicted in FIG. 7A.

Initially, the micro-organoids exposed to HFpEF-mimicking conditions were evaluated for the presence of hypertrophy and ultrastructural fibrosis, among the key hallmarks of HFpEF. Micro-organoids were stained for cardiac troponin T (cTnT) and DAPI to assess the cardiomyocyte cell density and viability (FIG. 7B). Following one week exposure to obesity-, hypertension-, and diabetes-mimicking conditions, organoids' size during maximal relaxation was increased by 0.03±0.001 mm² (P=0.003), 0.04±0.006 mm² (P=0.002), 0.05±0.005 mm² (P<0.001), respectively, when compared with 0.003±0.001 mm² increase in control RPMI. Notably, the combined exposure to all HFpEF-conditions resulted in the most substantial size augmentation: 0.07±0.004 mm² (P<0.001) compared to obesity (P<0.001), hypertension (P<0.001), diabetes (P=0.03), and RPMI (P<0.001) (FIG. 7C). The organoid fibrosis burden was quantified using automated image processing analysis for a Masson-Trichrome staining (FIG. 7D). Among the evaluated conditions, a significant increase in fibrosis burden was observed solemnly following exposure to the combined HFpEF-comorbidities, when compared with control group RPMI: 14.6±1.2% fibrosis burden for the HFpEF organoids vs. 6.8±1.5% in RPMI, P=0.001. Fibrosis burden for obesity, hypertension, and diabetes were 5.2±1.3% (P=0.96), 6.7±3.5% (P>0.99), and 9.0±1.1% (P=0.91), respectively compared with RPMI group (FIG. 7D, E).

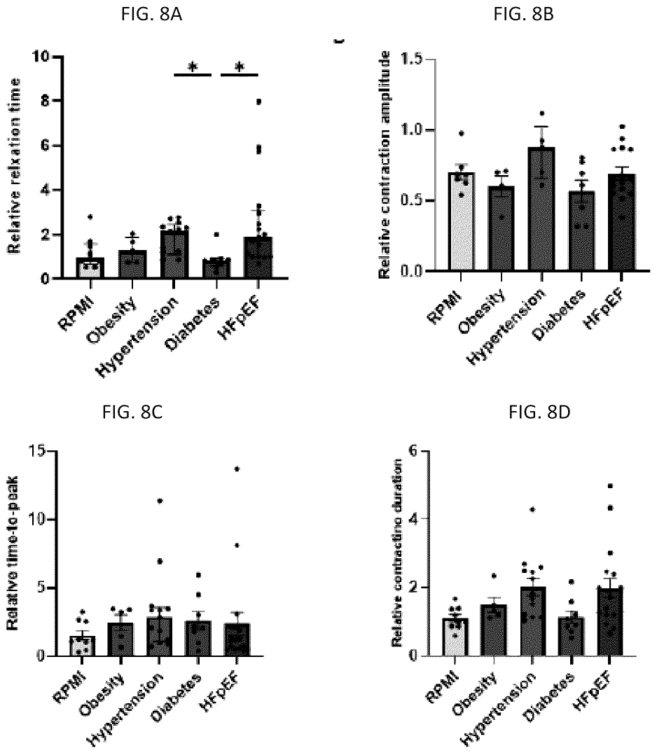

To further characterize our model, the micro-organoids contraction and relaxation were evaluated via microscopic video-based functional analysis (FIG. 8A-D). Compared to baseline (pre-treatment) data, the measured relaxation time was prolonged by 1.90-fold (IQR 1.02 to 3.12) following exposure to HFpEF-inducing conditions vs. 0.85-fold (IQR 0.60 to 0.93) for diabetic conditions (P=0.01) and a 0.95-fold (IQR 0.66 to 1.58) prolongation in RPMI (P=0.20). Exposure to hypertension-mimicking conditions resulted in a significant 2.16-fold (IQR 1.13 to 2.45) prolongation (P=0.028) compared to diabetes-conditions. Obesity-mimicking conditions resulted in a non-significant 1.30-fold (IQR 0.76 to 1.86) in relaxation time compared with RPMI (FIG. 8A). Contraction amplitude (a surrogate marker for the relative contractile forces) and time-to-peak contraction were not significantly altered by the different conditions (FIG. 8B-C). Analysis of contraction duration revealed overall significant differences among the groups (P=0.014), with numerical prolongation of contraction for both hypertension-mimicking and HFpEF-mimicking conditions but no post-hoc significant differences were detected (FIG. 8D).

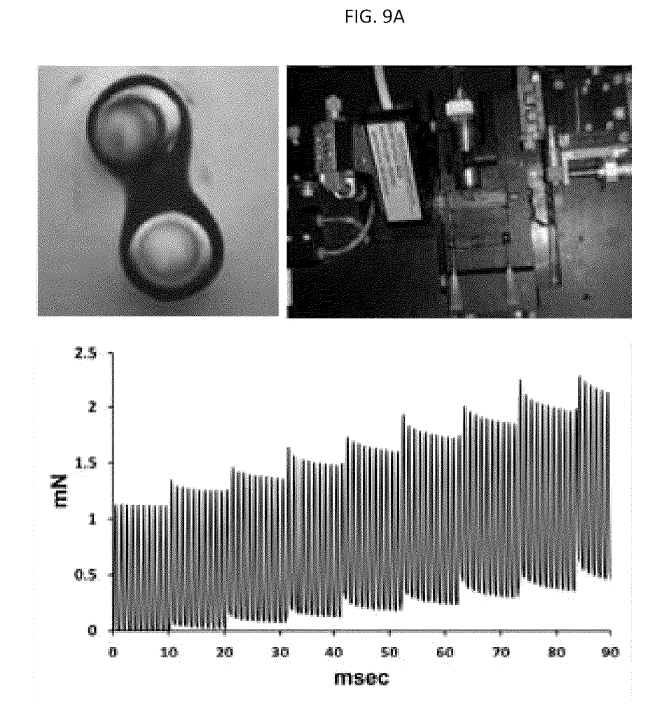

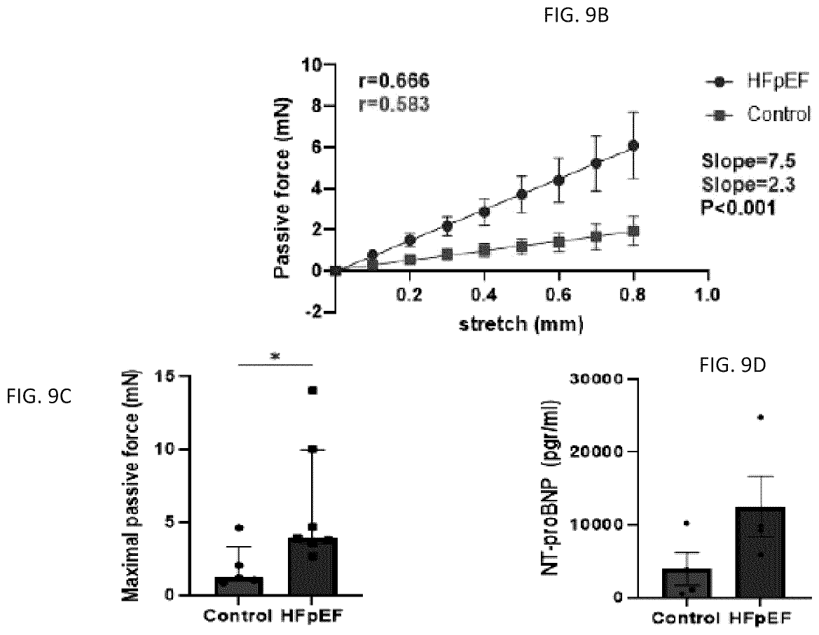

To further investigate the impact of HFpEF-inducing conditions on intricate cardiac physiological properties, length-force dynamics were assessed using larger, ring-shaped organoid model (2*10⁶ cells per organoid) (FIG. 9A). The human cardiac organoids (hCOs) platform facilitates the evaluation of the organoid's passive stiffness through preload stretching. In addition, for each preload length both passive and active forces are directly measured using a force transducer (FIG. 9A). Assessment of passive forces across escalating preload tissue lengths demonstrated a linear correlation between the two with Pearson's R correlation of 0.666 and 0.583 for HFpEF and control organoids, respectively. Importantly, the slope of the HFpEF regression model was significantly higher, compared to control, indicating elevated tissue stiffness: 7.52±1.08 mN/mm vs. 2.33±0.46 mN/mm, P<0.001 (FIG. 9B). The maximal passive force, at a tissue stretch of 0.8 mm was significantly higher in HFpEF-organoids: 3.93 mN (IQR 3.56 to 10.00) vs. 1.20 mN (IQR 0.95 to 3.33), respectively, P=0.03 (FIG. 9C). Furthermore, the mean NT-proBNP levels was numerically increased in HCOs following exposure to HFpEF-conditions and was measured as 12,414±4,200 μg/ml compared with 3,907±2,220 μg/ml in the control group, however this difference did not reach statistical significance (P=0.123, FIG. 9D).

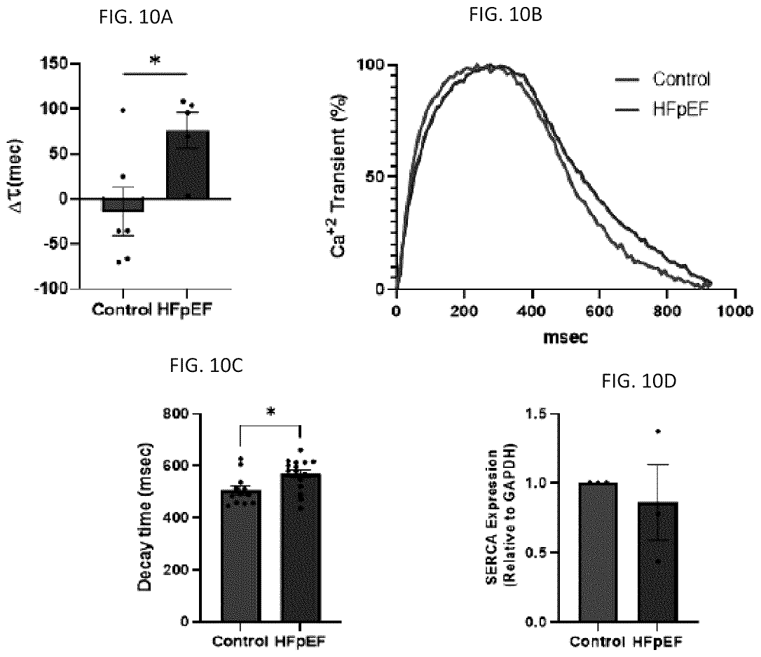

Diastolic relaxation properties were evaluated by measuring τ (tau), the relaxation time constant. With HFpEF-conditions, t was significantly prolonged compared to pre-exposure baseline by 76.27±19.44 msec, while 14.1±26.5 msec shortening was observed in the control group (P=0.026, FIG. 10A). To elucidate whether the prolongation of relaxation is associated with calcium handling abnormality, Ca⁺² transients in single cells were analyzed optically using a confocal microscopy (FIG. 10B). Exposure to HFpEF-conditions resulted in prolonged Ca⁺² clearance from the cytosol during diastole. A mean decay time of 567.2±16.2 msec was measured following HFpEF induction, compared to a significantly shorter, 505.5±16.9 msec for control (P=0.015, FIG. 10C). Real-time PCR analysis revealed that SERCA (sarcoplasmic/endoplasmic reticulum Ca⁺² ATPase) expression had a slightly lower expression for the HFpEF induced cardiomyocytes than the control, 0.86±0.27 (FIG. 10D).

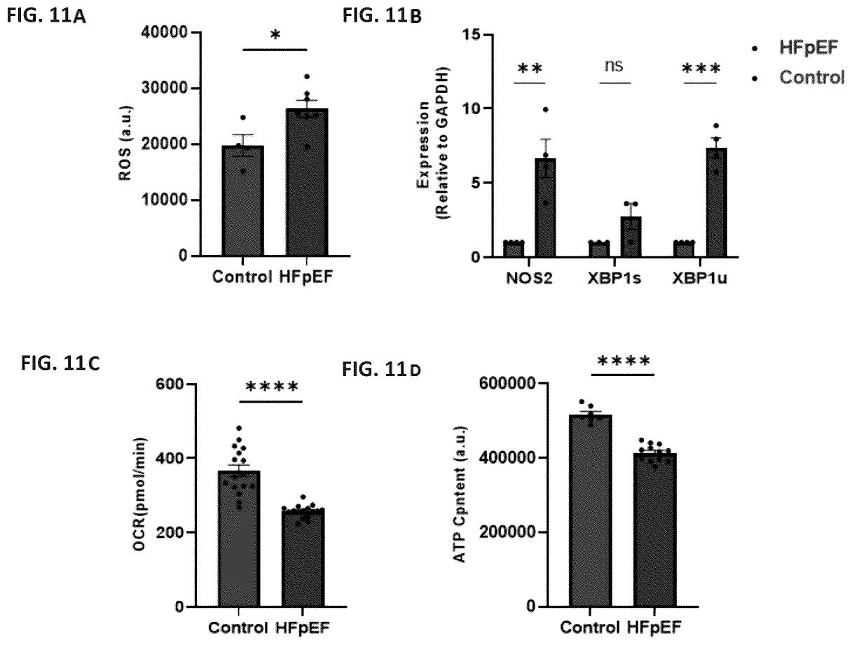

Finally, we examined the oxidative stress in the tissues, which is known as a mediator of several pathophysiological mechanisms associated with HFpEF including increased production of reactive oxygen species (ROS), mitochondrial dysfunction and impaired energetic status. Reactive oxygen species (ROS) content was significantly elevated in HFpEF-conditions: 26,350±1,494 a.u. in HFpEF tissues vs. 19,750±1,971 a.u. in control organoids media, (P=0.025, FIG. 11A). Real-time PCR analysis revealed a 6.65±1.3-fold increase in NOS2 expression (P=0.001), encoding to iNOS, a key factor in the pathophysiology of oxidative stress in HFpEF. Expression of the spliced form of Xbox-binding protein-1 (XBP1s), the active form, affecting as a transcription factor of ER-stress genes, was insignificantly elevated by 2.74±0.86 (P=0.407). The transcription of the inactive form, the un-spliced XBP1 (XBPlu) was also significantly increased by 7.35±0.68-fold (P<0.001, FIG. 11B). Study of mitochondrial function revealed a decreased oxygen consumption rate (OCR) of 256.9±4.4 μmol/min following exposure to HFpEF-conditions, compared to 366.7±15.8 μmol/min in the control (P<0.0001, FIG. 11C). ATP content was decreased accordingly, with 413,935±6,606 a.u. in HFpEF vs. 517,487±8,187 a.u. in control using CellTiter-Glo 2.0 ATP assay (P<0.001, FIG. 11D). Taken together, these findings demonstrate the induction of oxidative stress and metabolic dysfunction following exposure to HFpEF-conditions.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of generating a model for heart failure with preserved ejection fraction (HFpEF) comprising:
culturing human cardiomyocytes in a medium comprising at least one hypertension-associated hormone and at least one metabolic disease promoting agent, under conditions that bring about at least one of the following phenotypes:
(i) an increase in expression of collagen in said cardiomyocytes as compared to prior to said culturing;
(ii) an increase in relaxation time of said cardiomyocytes as compared to prior to said culturing;
(iii) an increase in production of NT-proBNP by said cardiomyocytes as compared to prior to the culturing; or
(iv) an increase in cardiomyocyte size as compared to prior to the culturing.

2. The method of claim 1, wherein said conditions bring about each of said phenotypes.

3. The method of claim 1, wherein said cardiomyocytes are comprised in a cardiac organoid.

4. The method of claim 3, wherein said cardiac organoid is a multicellular cardiac organoid.

5. The method of claim 4, wherein said multicellular cardiac organoid comprises endothelial cells, fibroblasts and smooth muscle cells.

6. The method of claim 1, wherein said human cardiomyocytes are derived ex vivo from pluripotent stem cells (PSCs).

7. The method of claim 6, wherein said pluripotent stem cells comprise induced PSCs.

8. The method of claim 1, wherein said human cardiomyocytes are mature cardiomyocytes.

9. The method of claim 8, further comprising maturing immature cardiomyocytes to generate the mature cardiomyocytes prior to said culturing.

10. The method of claim 9, wherein said maturing comprises culturing said immature cardiomyocytes in a medium comprising:
(i) an mTOR inhibitor; and
(ii) free fatty acids.

11. The method of claim 10, wherein said mTOR inhibitor comprises torin-1.

12. The method of claim 1, wherein said at least one hypertension-associated hormone comprises Angiotensin-II and/or Endothelin-1.

13. The method of claim 1, wherein said metabolic disease promoting agent comprises an obesity promoting agent and/or a diabetes promoting agent.

14. The method of claim 13, wherein said obesity promoting agent comprises fatty acids and inflammatory cytokines.

15. The method of claim 14, wherein said inflammatory cytokines are selected from the group consisting of IL-1β, IFN-γ, TNF-α and interleukin 1α.

16. The method of claim 13, wherein said diabetes promoting agent comprises glucose at a concentration above about 10 mM.

17. The method of claim 1, wherein said at least one hypertension-associated hormone comprises Angiotensin-II and/or Endothelin-1 and said at least one metabolic disease promoting agent comprises fatty acids, IL-1β, IFN-γ and glucose at a concentration above about 10 mM, and wherein said conditions comprise insulin depravation.

18. An HFpEF model generated according to claim 1.

19. A method of determining whether an active agent is useful for treating HFpEF comprising:
(a) contacting the agent with the model of claim 18,
(b) measuring at least one of the following characteristics of said cardiomyocytes:
(i) expression of collagen;
(ii) relaxation time;
(iii) production of NT-proBNP;
wherein a decrease in expression of collagen in said cardiomyocytes, a decrease in relaxation time, and/or a decrease in production of NT-proBNP as compared to prior to said contacting is indicative of an agent useful for treating HFpEF.
